(19) United States
(12) Patent Application Publication
(10) Pub. No.: US 2008/0131880 A1
(43) Pub. Date: Jun. 5, 2008

(54) PNA-DNA oligomers and methods of use thereof

(76) Inventors: Laura T. Bortolin, New Boston, NH (US); Christina M. Rudzinski, Belmont, MA (US); Amanda L. Stephens, Bedford, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/604,014

(22) Filed: Nov. 22, 2006

(51) Int. Cl. C12Q 1/68; C07H 21/04; C12P 19/34; G06G 7/48

(52) U.S. Cl. 435/6; 536/23.1; 435/91.2; 703/11

(57) ABSTRACT

Peptide nucleic acid (PNA)-deoxyribonucleic acid (DNA) oligomers and methods of using the PNA-DNA oligomers to detect and/or amplify a target nucleic acid in a sample are provided. The PNA-DNA oligomers of the invention are relatively insensitive to ionic concentration and many inhibitory proteins and, consequently, are particularly advantageous for direct use in environmental or challenging samples to detect nucleic acid, especially that of microorganisms, including food and water pathogens and/or bioterrorism agents. Methods are also provided for use of the PNA-DNA oligomers in applications including polymerase chain reaction, nucleic acid sequencing, mutation detection and as nucleic acid probes.

### GOVERNMENT SUPPORT

The invention was supported, in whole or in part, by grant F19628-00-C-0002 from the United States Air Force. The Government has certain rights in the invention.

### BACKGROUND OF THE INVENTION

Relatively short stretches of synthetic nucleic acid sequences (e.g., oligonucleotides) that bind specifically to another nucleic acid sequence are used in a number of applications. Typically, these short, synthetic oligonucleotides are primers or probes in applications that include, for example, nucleic acid amplification (e.g., polymerase chain reaction (PCR)), probing (e.g., northern or Southern blots or in situ hybridization), gene detection, sequencing, mutation detection and single nucleotide polymorphism (SNP) detection. The oligonucleotides are generally comprised of deoxyribonucleic acid (DNA), ribonucleic acid (RNA) and/or structural analogs of DNA or RNA designed to bind specifically to a particular region(s) of a nucleic acid.

The efficiency and/or success of the application in which these oligonucleotides are used is dependent on the efficiency at which they bind a nucleic acid sequence, and this binding efficiency is limited by a number of factors. These factors include the sequence of the oligonucleotides based on the target nucleic acid sequence and the conditions under which the oligonucleotides will have to bind the target nucleic acid. Although there is some flexibility in choosing the region at which a particular oligonucleotide will bind the nucleic acid sequence, the nucleotide sequence of the oligonucleotide is almost entirely dictated by sequence which is complementary to the nucleic acid sequence of this region. Achievement of the most stable binding of the oligonucleotides to the identified region of a target nucleic acid generally requires specific hybridization conditions. These conditions are typically sterile ones (e.g., those free of other proteins, contaminants and/or microbes), at a particular pH and salt concentration that is conducive to the specific application and/or components. The binding of the oligonucleotides also needs to take place under conditions which are free of proteins that inhibit their binding to the nucleic acid of interest. In the case of applications like PCR or sequencing, which require extension of oligonucleotides (e.g., primers) and progression of a polymerase (e.g., DNA polymerase) to form a complementary nucleic acid, this primer extension must also take place under conditions or in solutions free of proteins that inhibit the function and/or processivity of the DNA polymerase. Thus, in the absence of optimal conditions, the oligonucleotides currently used are subject to substantial inefficiencies in nucleic acid binding that ultimately influence their effectiveness in the applications in which they are employed. These inefficiencies also increase costs, as they result in a requirement for excess amounts of reagents (e.g., buffers, oligonucleotides, polymerases, cations) to off-set the inefficiencies in primer function.

Due to these limitations requiring optimal binding conditions for oligonucleotides, it is also difficult to detect a nucleic acid of interest in a sample without significant sample preparation and/or purification. Thus, nucleic acid detection and/or amplification generally cannot be performed in real time or in the field, from samples obtained directly from the environment without first purifying the sample to provide optimal conditions, for instance.

What is needed are oligonucleotides that bind a target nucleic acid more effectively and which are still functional, particularly in suboptimal conditions, thereby increasing the efficiency of the applications in which they are used and decreasing costs. Most preferable would be oligonucleotides that bind nucleic acid under a number of sample conditions, thus eliminating the loss in time inherent in the sample preparation that is currently required. Enhanced binding of these oligonucleotides would create an increased efficacy of various applications (e.g., PCR, sequencing, nucleic acid detection), resulting in better sensitivity in limits of detection and more rapid results.

### SUMMARY OF THE INVENTION

The present invention relates to peptide nucleic acid (PNA)-deoxyribonucleic acid (DNA) oligomers and methods of using the same. PNA binds more effectively and with higher affinity to nucleic acid than DNA or RNA, generally increasing the speed, efficiency and limits of detection of applications in which they are used. In particular, PNA-DNA oligomers are useful for nucleic acid detection under suboptimal conditions, that is, in samples that have not undergone purification (e.g., a non-pristine sample) and/or samples obtained directly from the environment (e.g., soil, water, air) or in a medical/forensic setting (e.g., blood, wound fluid, surface swab). In contrast, DNA oligomers (e.g., primers) are either highly ineffective in these conditions or fail altogether.

In particular, the present invention relates to a PNA-DNA oligomer comprising a PNA oligomer and a DNA oligomer wherein the PNA oligomer and DNA oligomer are covalently linked by a C₆ amino linker having the formula:

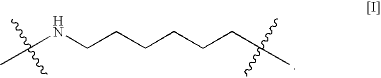

Further, a method of using a PNA-DNA oligomer is provided in a method for detecting the presence of a target nucleic acid in a sample. The method comprises combining a peptide nucleic acid (PNA)-deoxyribonucleic acid (DNA) oligomer, a labeled probe, a DNA polymerase having exonuclease activity, unlabeled deoxyribonucleotide triphosphates (dNTPs) and the sample, thereby forming a combination. The combination is maintained under conditions suitable for extending the PNA-DNA oligomer in the presence of the target nucleic acid. Thus, the PNA-DNA oligomer and labeled probed are annealed to the target nucleic acid, wherein the labeled probe anneals to the target nucleic acid downstream of the PNA-DNA oligomer. Under the conditions suitable for extending the PNA-DNA oligomer, the DNA polymerase extends the PNA-DNA oligomer annealed to the target nucleic acid in the presence of the unlabeled dNTPs, thereby forming a full-length unlabeled nucleic acid product. As the PNA-DNA oligomer annealed to the target nucleic acid is extended by the DNA polymerase, the exonuclease activity of the DNA polymerase degrades the labeled probe also annealed to the target nucleic acid, degradation of the probe resulting in emission of a detectable signal. The combination is analyzed for emission of this detectable signal, where the emission of the detectable signal indicates the presence of the target nucleic acid in the sample.

In accordance with one aspect of the method, the probe is fluorescently labeled in a fluorescent resonance energy (FRET) format in which a fluorescent, high energy dye (donor) is on one end of the probe and quencher low energy dye (acceptor) is on the other end of the probe. When the probe is intact and the two dyes are in close proximity, the energy from the fluorescent, high energy donor is transferred to the low energy acceptor such that fluorescence of the donor dye is quenched. Upon cleavage of the probe (e.g., by the DNA polymerase), the two dyes are no longer in close proximity and fluorescence of the high energy dye is no longer quenched, resulting in emission of a detectable fluorescent signal. In a particular embodiment, the method can further comprise amplifying the full-length unlabeled nucleic acid product, thereby amplifying the detectable signal. In another embodiment, the PNA-DNA oligomer is comprised of a PNA oligomer and a DNA oligomer, wherein the PNA and DNA are covalently linked by a linker having the formula:

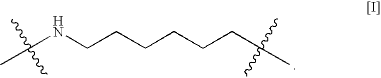

In yet a further embodiment, the sample in which the target nucleic acid is detected can be a biological sample (e.g., blood, saliva, wound fluid), environmental sample (e.g., soil, water, air), contaminated sample, suboptimal sample (e.g., challenging, high/low ionic strength) or a purified sample or pristine sample (e.g., nucleic acid isolated and/or purified, e.g., using a commercial kit (e.g., QIAGEN®) or maxi-preparation), and combinations thereof. In a particular embodiment, the emission of the detectable signal indicates the presence of a target nucleic acid of a microorganism (e.g., B. anthracis).

A method for detecting a target ribonucleic acid (RNA) in a sample is also provided. The method comprises combining a first peptide nucleic acid (PNA)-deoxyribonucleic acid (DNA) oligomer, a reverse transcriptase enzyme, unlabeled deoxyribonucleotide phosphates (dNTPs) and the sample, thereby forming a first combination. This combination is maintained under conditions suitable for extending first PNA-DNA oligomer in the presence of the target RNA that is in the sample, wherein the first PNA-DNA oligomer anneals to the target RNA and the reverse transcriptase enzyme extends the first PNA-DNA oligomer, thereby forming an unlabeled cDNA product. The unlabeled cDNA product that forms is combined with a second PNA-DNA oligomer, a labeled probe and a DNA polymerase having exonuclease activity, thereby forming a second combination. The second combination is maintained under conditions suitable for extending the second PNA-DNA oligomer and the labeled probe in the presence of the unlabeled cDNA product, wherein the second PNA-DNA oligomer and the labeled probe anneal to the unlabeled cDNA product and wherein the labeled probe anneals to the unlabeled cDNA product downstream of the second PNA-DNA oligomer. In the second combination, the second PNA-DNA oligomer is extended with the DNA polymerase having exonuclease activity in the presence of unlabeled dNTPs, thereby forming a full-length unlabeled DNA product. In the process of extending the second PNA-DNA oligomer annealed to the unlabeled cDNA product, the exonuclease activity of the DNA polymerase degrades the labeled probe also annealed to the unlabeled cDNA product, thereby resulting in emission of a detectable signal from the probe. The second combination is analyzed for emission of this detectable signal, wherein emission of the detectable signal indicates the presence of the target RNA in the sample.

Yet another method for detecting a target nucleic acid in a sample is provided, the method comprising combining a peptide nucleic acid (PNA)-DNA oligomer, DNA polymerase, unlabeled deoxyribonucleotide triphosphates (dNTPs) and the sample, thereby forming a combination. The method comprises maintaining the combination under conditions suitable for extending the PNA-DNA oligomer in the presence of the target nucleic acid in the sample, wherein the PNA-DNA oligomer anneals to the target nucleic acid and the DNA polymerase extends the PNA-DNA oligomer in the presence of the unlabeled dNTPs, thereby forming a full-length unlabeled target nucleic acid product. The full-length unlabeled target nucleic acid product is amplified by: i) denaturing the full-length unlabeled target nucleic acid product, ii) maintaining the PNA-DNA oligomer, a DNA polymerase, a reverse complementary primer and the unlabeled target nucleic acid product under conditions for extending the PNA-DNA oligomer and the reverse complementary primer in the presence of the denatured full-length unlabeled target nucleic acid product. The PNA-DNA oligomer and the reverse complementary primer anneal to the denatured full-length unlabeled target nucleic acid product and the DNA polymerase extends the PNA-DNA oligomer and the reverse complementary primer in the presence of unlabeled dNTPs, thereby forming a full-length target nucleic acid product. Step i) and ii) are repeated one or more times, thereby producing one or more full-length-unlabeled target nucleic acid products. The one or more full-length unlabeled target nucleic acid products formed are detected, where the presence of one or more full-length target nucleic acid products indicates the presence of the target nucleic acid in the sample. The one or more unlabeled target nucleic acid products can be detected by a DNA-intercalating agent or dye, or with a labeled probe.

A method of amplifying a target nucleic acid is also provided. The method comprises combining a peptide nucleic acid (PNA)-DNA oligomer, a reverse complementary primer, a labeled probe, a DNA polymerase having exonuclease activity, unlabeled deoxyribonucleotide triphosphates (dNTPs) and the target nucleic acid, thereby forming a combination. In one embodiment, the target nucleic acid (e.g., a double-stranded target nucleic acid) is denatured. The combination is maintained under conditions suitable for extending said PNA-DNA oligomer in the presence of the target nucleic acid. The PNA-DNA oligomer, reverse complementary primer and labeled probe anneal to the target nucleic acid, where the labeled probe anneals to the target nucleic acid downstream of the PNA-DNA oligomer and, under conditions suitable for extension of the PNA-DNA oligomer, the DNA polymerase extends the PNA-DNA oligomer annealed to the target nucleic acid in the presence of the unlabeled dNTPs, thereby forming full-length unlabeled target nucleic acid product. The exonuclease activity of the DNA polymerase degrades the labeled probe annealed to the target nucleic acid during extension of the PNA-DNA oligomer annealed to the target nucleic acid, which results in the emission of a detectable signal. The full-length unlabeled target nucleic acid product formed is denatured. The above steps (i.e., PNA-DNA oligomer and reverse complementary primer annealing and extension to form full-length unlabeled target nucleic acid product and denaturation of the full-length unlabeled target nucleic acid product) are repeated one or more times, thereby forming one or more unlabeled target nucleic acid products. This repetition results in amplification of the target nucleic acid. The method can also further comprise detecting the emission of the detectable signal, so that amplification of the target nucleic acid is detected in real time. In accordance with another aspect of the method, the PNA-DNA oligomer comprises a PNA oligomer and a DNA oligomer covalently linked by a C₆ amino linker having the formula:

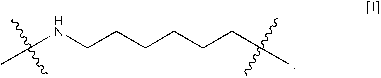

Another method of amplifying a target nucleic acid is also provided. The method comprises combining a peptide nucleic acid (PNA)-deoxyribonucleic (DNA) oligomer, a reverse complementary primer, a DNA polymerase, unlabeled deoxyribonucleotide triphosphates (dNTPs) and the target nucleic acid and to form a combination. The method further comprises maintaining the combination under conditions suitable for extending the PNA-DNA oligomer and the reverse complementary primer in the presence of the target nucleic acid. The annealed PNA-DNA oligomer and reverse complementary primer are extended with the DNA polymerase in the presence of the unlabeled dNTPs, thereby forming full-length unlabeled target nucleic acid product. The full-length unlabeled target nucleic acid product is denatured and the above processes repeated (i.e., PNA-DNA oligomer and reverse complementary primer extension to form full-length unlabeled target nucleic acid product and denaturation of full-length unlabeled target nucleic acid product formed) one or more times, thereby forming one or more full-length unlabeled target nucleic acid products and thereby amplifying the target nucleic acid. In accordance with one aspect of the method, the target nucleic acid is denatured prior to the annealing of the PNA-DNA oligomer and the reverse complementary primer to the target nucleic acid. In accordance with another aspect of the method, the PNA-DNA oligomer comprises a PNA oligomer and a DNA oligomer covalently linked by a C₆ amino linker having the formula:

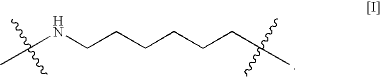

In addition, a method of probing a target nucleic acid is also provided. The method comprises hybridizing to the target nucleic acid an unlabeled peptide nucleic acid (PNA)-DNA oligomer comprising at least one PNA oligomer and at least one DNA oligomer, wherein the at least one PNA oligomer and at least one DNA oligomer are covalently linked by a linker selected from the group consisting of a C₆ amino linker having the formula:

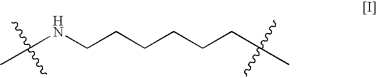

and a C₅ carboxy linker having the formula:

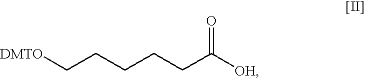

where DMTO is 3,5-dimethyl-1,2,4-trioxolane. Hybridization of the unlabeled PNA-DNA oligomer is detected. In one embodiment, the hybridization of the unlabeled PNA-DNA oligomer is detected by the cyanine dye 3,3′-diethylthiadicarbocyanine iodide (DiSc₂(5)). In a particular embodiment, the method is used to detect one or more nucleic acid changes including a mutation to, amplification of, addition to, insertion in and deletion in the target nucleic acid.

In addition, a method for designing a PNA-DNA oligomer that binds a target nucleic acid is provided (see also FIG. 7). The method comprises obtaining the sequence of the target nucleic acid and determining a complementary PNA-DNA oligomer sequence for a region on the target nucleic acid, thereby identifying a potential PNA-DNA oligomer. The potential PNA-DNA oligomer is then accepted or rejected in a method comprising: calculating the percent of guanine (G) and cytosine (C) nucleotides in the potential PNA-DNA oligomer, wherein if the percent of G and C nucleotides is between about 30% and about 80%, then the potential PNA-DNA oligomer is accepted. The melting temperature of the potential PNA-DNA oligomer is also calculated, wherein if the melting temperature is between about 54° C. and about 64° C., then the potential PNA-DNA oligomer is accepted. The number of contiguous adenine (A), contiguous thymine (T), contiguous guanine (G) and contiguous cytosine (C) nucleotides in the potential PNA-DNA oligomer is determined, wherein if there are less than: (a) four contiguous A nucleotides, (b) four contiguous T nucleotides, (c) three contiguous C nucleotides and (d) three contiguous G nucleotides, then the potential PNA-DNA oligomer is accepted. For the PNA oligomer portion of the potential PNA-DNA oligomer, the method further comprises calculating the percent of adenine (A) and guanine (G) nucleotides, thereby determining the purine content of the potential PNA-DNA oligomer, wherein if the percent of A and G nucleotides is less than or equal to about 60%, then the potential PNA-DNA oligomer is accepted. The percent of guanine (G) and cytosine (C) nucleotides in the PNA oligomer portion of the potential PNA-DNA oligomer is also calculated, wherein if the percent of G and C nucleotides is between about 30% and about 80%, then the potential PNA-DNA oligomer is accepted. The method also comprises calculating the melting temperature of the PNA oligomer portion of the potential PNA-DNA oligomer, wherein if the melting temperature is between about 9° C. and about 15° C., then the potential PNA-DNA oligomer is accepted. In addition, the number of contiguous G and C nucleotides in the PNA oligomer portion of the potential PNA-DNA oligomer is determined, wherein if there are less than three contiguous G or three contiguous C nucleotides, then the PNA-DNA oligomer is accepted. In one embodiment, the PNA-DNA oligomer designed can be a forward primer or a reverse primer.

In accordance with one aspect of the method, the method further comprises designing a probe by determining a probe-binding region on the target nucleic acid between 1 and 5 nucleotides 3′ to the region that the PNA-DNA oligomer binds to the target nucleic acid and determining a complementary nucleotide sequence for the probe-binding region, thereby identifying a potential probe. The potential probe is accepted or rejected in a method comprising calculating the percent of cytosine (C) and guanine (G) nucleotides in the potential probe, wherein if the percent of C and G nucleotides is between about 30% and about 80%, then the potential probe is accepted. The melting temperature for the potential probe is also calculated, wherein if the melting temperature is between about 65° C. and about 85° C. then the potential probe is accepted. The number of contiguous adenine (A), contiguous thymine (T), contiguous guanine (G) and contiguous cytosine (C) nucleotides in the potential probe is also determined in the method, wherein if there are less than: (a) four contiguous A nucleotides, (b) four contiguous T nucleotides, (c) three contiguous C nucleotides and (d) three contiguous G nucleotides, then the potential probe is accepted. The first nucleotide base of the potential probe is also identified, wherein if the first nucleotide base is an adenine (A), thymine (T) or a cytosine (C), then the potential probe is accepted.

The present invention also relates to computer products comprising computer useable medium including a computer readable program that, when executed, causes the computer to design an acceptable PNA-DNA oligomer and/or a probe based on the criteria recited in the above-mentioned methods.

The PNA-DNA oligomers of the present invention overcome many of the limitations of other nucleic acid primers and probes in that the PNA-DNA oligomers have a greater binding affinity for nucleic acid, can bind to a target nucleic acid under a number of conditions, including in samples that have undergone little to no preparation and/or purification, and are robust, in that they are not recognized and degraded by cleavage enzymes (e.g., DNases, RNases, proteases or proteinases). Thus, the PNA-DNA oligomers can be used efficiently in and improve the effectiveness of methods that detect and/or amplify a target nucleic acid or a particular region of a target nucleic acid. In particular, the PNA-DNA oligomers can be used to rapidly and reliably detect the nucleic acid of microorganisms, especially pathogens or bioterrorism agents, in the environment (e.g., water, soil, air) or on food products.

### BRIEF DESCRIPTION OF THE DRAWINGS

The foregoing will be apparent from the following more particular description of example embodiments of the invention, as illustrated in the accompanying drawings. The drawings are not necessarily to scale, emphasis instead being placed upon illustrating embodiments of the present invention.

FIG. 1 is an illustration of a PNA molecule depicted like peptides with the N-terminus of at the top position and the C-terminus at the bottom position. B=a nucleotide base (e.g., A, T, C or G).

FIG. 2 is an illustration of a protein molecule, PNA molecule and DNA molecule. The PNA and DNA are shown in hybridized, double-stranded configuration.

FIG. 3 is a schematic illustrating a PNA-DNA oligomer bound to target DNA.

FIG. 4 is a schematic illustrating the linker C₆ amino between PNA and DNA.

FIG. 5 is a schematic illustrating binding of two PNA-DNA oligomers and a fluorescently labeled DNA probe to a denatured double-stranded DNA template. The DNA probe is labeled in a FRET format with a high energy fluorescent moiety ( [symbol] ) on the 5′ end of the probe and a low energy moiety ( [symbol] ) on the 3′ end of the probe. The PNA portion (▪) of the PNA-DNA oligomer is located on the 5′ end of the oligomer and binds to the 3′ end of both strands of the DNA template ( [symbol] ).

FIG. 6 is a schematic illustrating the amplification of target DNA using two PNA-DNA oligomers.

FIG. 7 is a flow diagram illustrating the process for design of a PNA-DNA oligomer (see Example 3).

FIG. 8 is a flow diagram illustrating the process for design of a probe (see Example 3).

FIG. 9 illustrates a computer network or similar digital processing environment in which a computer program product for design of a PNA-DNA oligomer or a probe can be implemented.

FIG. 10 is a diagram illustrating the internal structure of a computer in the computer system of FIG. 9.

FIG. 11 is a graph illustrating real-time PCR of B. anthracis DNA by PNA-DNA oligomers and DNA primers under standard conditions (see Example 2).

FIG. 12 is a graph illustrating real-time PCR of B. anthracis DNA by PNA-DNA oligomers and DNA primers in soil samples (see Example 2).

FIG. 13 is a graph illustrating real-time PCR of B. anthracis DNA by PNA-DNA oligomers and DNA primers blood samples (see Example 2).

FIGS. 14A-14B list the sequences of different PNA-DNA oligomers tested that were specific for the detection of B. anthracis protective antigen gene (PAG). PAG refers to a first set of oligomers (SEQ ID NOs. 1-11) (FIG. 14A). PAGII refers to a second set of oligomers (SEQ ID NOs. 12-26) (FIG. 14B) (see also Example 2).

FIG. 15 is a table illustrating the results of real-time PCR of B. anthracis (BA) protective antigen gene (PAG) using different PNA-DNA oligomers and DNA concentrations under standard conditions (see Example 2). PPS=primer/probe set; CT=cycle threshold.

FIG. 16 is a table illustrating the results of real-time PCR of B. anthracis protective antigen gene (PAG) using different PNA-DNA oligomers in various dilutions of whole blood (see Example 2). PPS=primer/probe set; CT=cycle threshold.

FIG. 17 is a table illustrating the results of real-time PCR of B. anthracis protective antigen gene (PAG) using different PNA-DNA oligomers in various dilutions of electrostatic collector fluid (see Example 2). PPS=primer/probe set; CT=cycle threshold.

FIGS. 18A and 18B are tables illustrating the results of real-time PCR of B. anthracis protective antigen gene using different PNA-DNA oligomers in various dilutions of soil (see Example 2).

FIG. 19A is a table illustrating the results of experiments that determined the design constraints for PAG PNA-DNA oligomers.

FIG. 19B is a table illustrating the results of experiments that determined the design constraints for PAGII PNA-DNA oligomers.

### DETAILED DESCRIPTION OF THE INVENTION

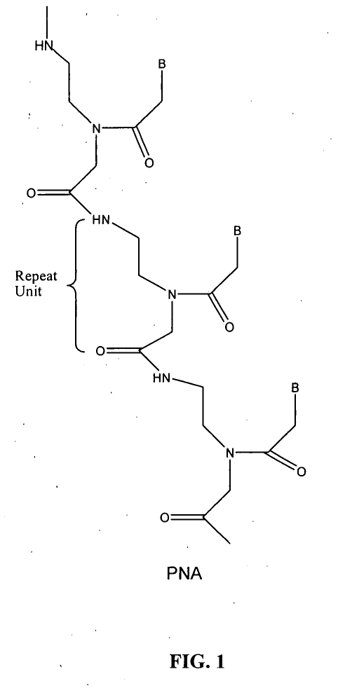

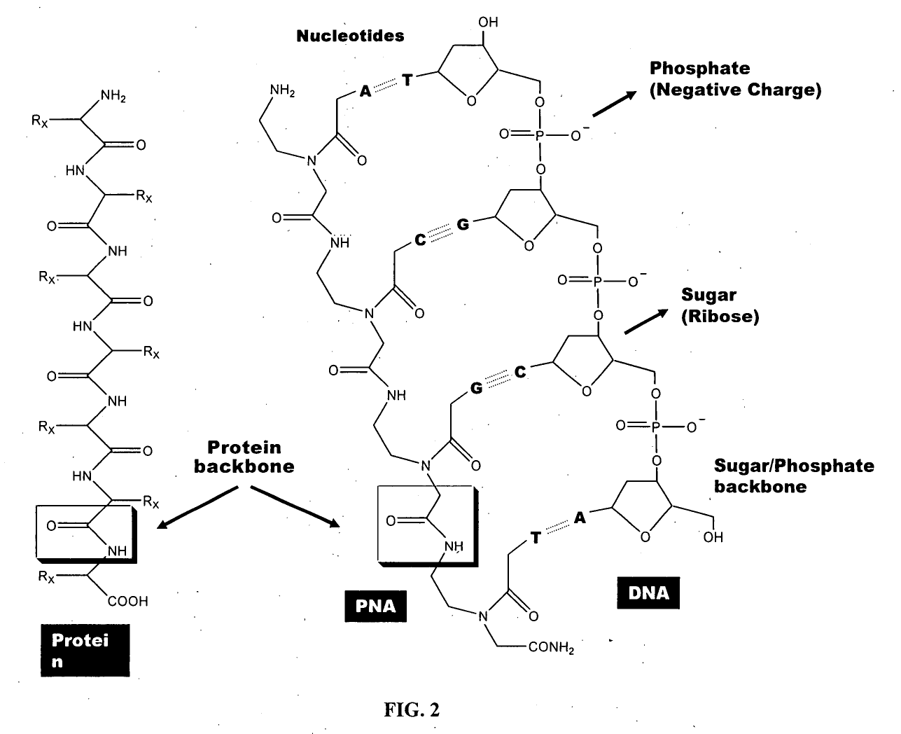

The present invention relates to peptide nucleic acid (PNA)-deoxyribonucleic acid (DNA) oligomers and their use in various applications that relate to identifying, amplifying, probing, sequencing, inhibiting production of gene products and/or ascertaining mutations in nucleic acids. Peptide nucleic acid (PNA) is a structural nucleic acid (e.g., DNA or RNA) analog able to bind to a complementary nucleic acid sequence. PNA has an acyclic, achiral, uncharged backbone composed of N-(2-amino-ethyl)-glycine units linked by peptide bonds. Purines and pyrimidines are attached to this backbone by methylene carbonyl linkages (see FIG. 1). Thus, as used herein, “peptide nucleic acid base” refers to a unit of PNA that binds to a complementary nucleotide (e.g., DNA, RNA) base (e.g., adenine, thymine, uracil, guanine, cytosine). The lack of charge of the PNA backbone results in stronger binding (e.g., increased melting temperature) between PNA and nucleic acid due to a lack of charge repulsion between the two (see FIG. 2). In addition, PNA has several other advantages over natural oligonucleotides including: better binding specificity, increased stability, resistance to enzymatic cleavage (by e.g., proteinases, DNases) due to a lack of recognition by the enzymes and membrane permeability due to the neutral backbone of PNA. These advantages are conferred on the PNA-DNA oligomers of the invention.

With the aforementioned advantages in nucleic acid binding, the PNA-DNA oligomers can be used as, for example, primers in applications like polymerase chain reaction (PCR), under conditions in which traditional DNA-only primers are mostly, if not completely, ineffective. For instance, as described herein, in PCR, the PNA-DNA oligomers successfully amplify DNA from samples like soil, blood and electrostatic fluid, these samples not having undergone any, or any appreciable, purification. Normally such samples would need to be purified for applications using traditional DNA primers and/or probes, otherwise the primer and/or probe would fail to work or yield poor results. Thus, these otherwise sub-optimal samples are now suitable for applications using the PNA-DNA oligomers of the present invention. As described herein, the samples can have characteristics like high ionic strength (e.g., high salt concentration), non-neutral pH and/or proteins inhibitory to nucleic acid binding and/or extension (e.g., inhibitory to primer-DNA binding and/or DNA polymerase nucleic acid template extension). Thus, in these and other applications, the PNA-DNA oligomers described herein can be used in sub-optimal (e.g., non-pristine) conditions and, further, in non-laboratory type settings (e.g., in the field/environment, at a point-of-care). In addition, the PNA-DNA oligomers of the present invention can also be used in pristine conditions.

Accordingly, the present invention relates to a PNA-DNA oligomer comprising a PNA oligomer and a DNA oligomer, wherein the PNA oligomer and DNA oligomer are covalently linked. In a particular embodiment the linker is small, simple in structure and flexible to less hinder binding of the PNA-DNA oligomer to the nucleic acid. Thus, in one embodiment, the PNA oligomer and DNA oligomer are covalently linked by a C₆ amino linker having the formula:

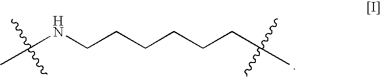

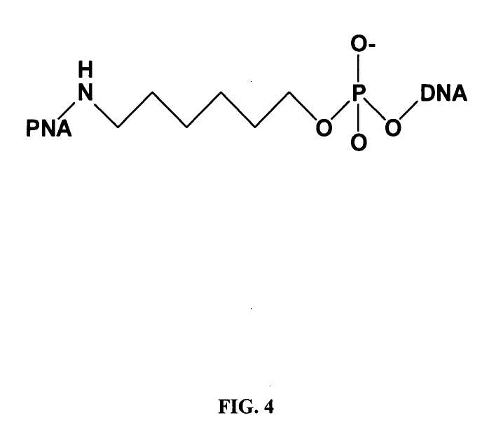

(See, e.g., FIG. 4). The PNA-DNA oligomers bind to nucleic acid (e.g., DNA, RNA) with higher binding affinity than DNA oligonucleotides alone, are resistant to degradation and have an ability to bind nucleic acid in a sample, independent of the ionic strength and purity of the sample. These properties increase the speed and efficacy of applications in which the PNA-DNA oligomers are used.

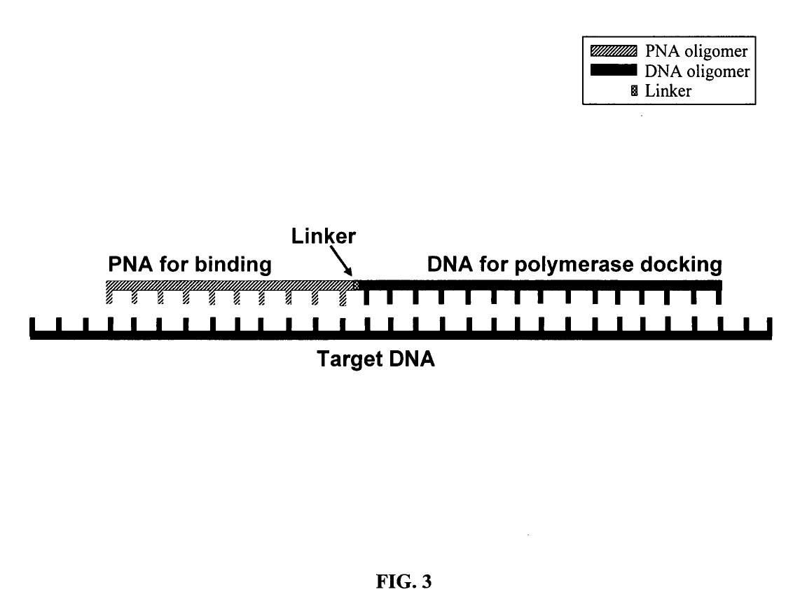

The PNA oligomer of the PNA-DNA oligomers of the invention, also referred to herein as primers or oligonucleotides, can be synthesized from monomers (see, e.g., Koch et al., J. Pept. Res. 49(1):80-88, 1997) or the oligonucleotide analogs can be obtained commercially (from, e.g., Metkinen Chemistry, Kuusisto, Finland) and synthesized on automated synthesizers (e.g., ABI 443A, 394, Expedite, Applied Biosystems). Alternatively, the PNA-DNA oligomers can also be synthesized commercially (by, e.g., Eurogentec, San Diego, Calif., USA, Biosynthesis, Inc., Lewisville, Tex., USA, ASM Research Chemicals, Bomlitz, Germany). During or after the synthesis of the PNA oligomer and DNA oligomer, the two are linked covalently by the linker C₆ amino having the formula [I] above. The appropriate linkage between the PNA oligomer and the DNA oligomer is needed for the design and efficacy of the PNA-DNA oligomer in various applications. The C₆ amino linker is small, essentially equivalent to the size of one nucleotide base. Thus, its structure is simple and more akin to that of a natural oligonucleotide (e.g., DNA oligonucleotide), which causes less steric hindrance to nucleic acid-binding by the PNA-DNA oligomer. In addition, the C₆ amino linker likely has a significant degree of flexibility. It is believed that the size, simplicity and flexibility of the C₆ amino linker means that, unlike the PNA oligomer and DNA oligomer, it does not bind the nucleic acid (e.g., the linker is excluded from nucleic acid binding) (see FIG. 3). That is, the linker would not interfere with PNA-DNA oligomer nucleic acid-binding or with any other reactions that occur in applications in which the PNA-DNA oligomers are used (e.g., polymerase chain reaction (PCR)).

In a particular embodiment, the PNA oligomer is coupled to the C₆ amino linker through an amide bond on the 5′ end of the linker and the phosphate bond of the DNA oligomer is coupled to the 3′ end of the linker, thereby forming the PNA-DNA oligomer (see FIG. 4). In another embodiment, a C₅ carboxy linker having the formula:

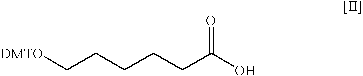

(where DMTO is 3,5-dimethyl-1,2,4-trioxolane) can be used to attach the DNA oligomer to the PNA oligomer such that the DNA oligomer is coupled to the 5′ end of the linker through a phosphate bond and the PNA oligomer is coupled to a carboxyl bond on the 3′ end of the linker (e.g., a DNA-PNA oligomer). Different orientations of the PNA oligomer and DNA oligomer may be preferable depending on the application in which the PNA-DNA oligomer is to be used. For example, in applications like PCR or sequencing, which require enzyme (e.g., DNA polymerase) binding and extension of the PNA-DNA oligomer, the PNA oligomer needs to be 5′ to the DNA oligomer which, unlike PNA, supports nucleotide extension by DNA polymerase. However, in applications in which the PNA-DNA oligomer acts as a nucleic acid probe (e.g., in situ hybridization, northern blot, Southern blot, mutation analysis), the PNA oligomer and DNA oligomer can be linked in either orientation, with the chosen orientation based on that which results in optimal binding of the PNA-DNA oligomer to the region of interest on a particular nucleic acid. The orientation in which the PNA is in the 5′ position of the PNA-DNA oligomer generally results in more stable nucleic acid binding.

The PNA oligomer can be of any length sufficient to enhance binding of the PNA-DNA oligomer to a nucleic acid. Due to the higher binding affinity of PNA for nucleic acid, it is not necessary to design PNA oligomers to be as long as traditional oliogonucleotide oligomers (e.g., traditionally 20-25 bases). In particular applications, it may be necessary for the PNA oligomer to have few enough bases to allow the PNA-DNA oligomer to dissociate from a nucleic acid. Thus, in one embodiment, the PNA oligomer is comprised of at least one peptide nucleic acid base and, in another embodiment, can be comprised of about 4 to about 7 such bases. The peptide nucleic acid bases themselves and the PNA oligomers can be synthesized on automated machines and both the PNAs, and the PNA oligomers are commercially available (e.g., Metkinen Chemistry, ASM Research Chemicals, Biosynthesis, Inc., Eurogentec).

Similarly, the DNA oligomer portion of the PNA-DNA oligomer can be of any length so long as the DNA oligomer does not decrease overall binding affinity and/or efficiency of the PNA-DNA oligomer. The length and/or composition of the DNA oligomer is limited by potential/predicted intra- (complementarity within the DNA oligomer) or inter-(complementarity within the PNA-DNA oligomer) molecular binding that could result in the formation of secondary structure (e.g., loops, hairpins) and prevent binding of the PNA-DNA oligomer to a target nucleic acid (e.g., a nucleic acid of interest in a particular application).

Like traditional DNA primers and probes, several parameters should be considered in identifying the sequence of both the PNA and DNA portions of the PNA-DNA oligomer for binding to a target nucleic acid in a particular application. Beyond the length of the PNA-DNA oligomer, parameters like melting temperature, guanine (G) and cytosine (C) content, stretches of the same nucleotide base, PNA purine content and PNA and/or DNA geometry should be considered for optimal performance and/or binding of the PNA-DNA oligomer. As described herein, Applicants have defined these parameters. Thus, in a particular embodiment, the PNA-DNA oligomer has a purine content of less than about 60%, a GC content of between about 30% to about 80%, a melting temperature of between about 54° C. and about 64° C., single nucleotide repeats of adenine (A) and thymine (T) of less than 4 bases and single nucleotide repeats of G and C of less than 3 bases.

The present invention also relates to the use of the PNA-DNA oligomer(s) of the invention in various applications such as to detect or amplify a nucleic acid of interest, detect the presence of a particular change or mutation in a nucleic acid, determine the sequence of a particular nucleic acid or detect or inhibit the expression of a gene. These applications encompass polymerase chain reaction (PCR), including reverse-transcriptase PCR(RT-PCR) and real-time or quantitative PCR (QPCR), DNA sequencing, transcriptional expression mapping and profiling (e.g., microarrays and gene chips), northern and Southern blotting, antisense agents, disease diagnostics in man and other animals, genotyping of humans, animals (e.g., purebred horses, dogs or animal stocks) and plants (e.g., crops to ensure genetic modifications or a lack thereof) and determining antibiotic resistance in humans, animals and/or microorganisms. As the person of skill in the art will appreciate, the steps and/or components of the methods described herein can be performed simultaneously and/or sequentially, as appropriate.

Thus, one embodiment of the present invention relates to a method for detecting the presence of a target nucleic acid in a sample. The method comprises combining a peptide nucleic acid (PNA)-deoxyribonucleic acid (DNA) oligomer, a labeled probe, a DNA polymerase having exonuclease activity, unlabeled deoxyribonucleotide triphosphates (dNTPs) and the sample, thereby forming a combination. As one of skill in the art will appreciate, one or more of components can be added simultaneously or sequentially in the methods provided herein. The combination is then maintained under conditions suitable for extending the PNA-DNA oligomer in the presence of the target nucleic acid. Thus, the PNA-DNA oligomer and labeled probed are annealed to the target nucleic acid, where the labeled probe anneals to the target nucleic acid downstream of the PNA-DNA oligomer. Under conditions suitable for extending the PNA-DNA oligomer, the DNA polymerase extends the PNA-DNA oligomer annealed to the target nucleic acid in the presence of the unlabeled dNTPs, thereby forming full-length unlabeled nucleic acid product. As the PNA-DNA oligomer annealed to the target nucleic acid is extended by the DNA polymerase, the exonuclease activity of the DNA polymerase degrades the labeled probe also annealed to the target degradation of the probe resulting in emission of a detectable signal. The combination is analyzed for emission of this detectable signal, where the emission of the detectable signal indicates the presence of the target nucleic acid in the sample. In addition, one or more target nucleic acids can be detected in a sample using one or more PNA-DNA oligomers, each oligomer specific for each of the one or more target nucleic acids in the sample along with the associated sequence-specific labeled probe. In this embodiment, the probes would be differentially labeled in order to identify the presence of each unique target nucleic acid in the sample.

The target nucleic acid (e.g., a nucleic acid of interest and/or desired to be detected/identified and/or amplified) bound by the PNA-DNA oligomer can by any double- and/or single-stranded nucleic acid or nucleic acid analog including deoxyribonucleic acid (DNA), genomic DNA, mitochondrial DNA, synthetic DNA, genetically-engineered DNA, plasmid or vector DNA, chromosomal DNA, DNA fragments, polymerase chain reaction (PCR) amplicon DNA, transposon DNA, viral DNA, ribonucleic acid (RNA), viral RNA, ribosomal RNA, synthetic RNA and combinations thereof. Depending on the particular application, the target nucleic acid can be of any length, from several nucleotide bases to millions of nucleotide bases (megabases) and is at least sufficiently long to be bound by a PNA-DNA oligomer. The sequence of the target nucleic acid can be known or partially known or unknown. The amount of sequence that is known need only be enough to accomplish the particular application (e.g., PCR, sequencing), as assessed by the skilled artisan.

Due to the enhanced binding affinity and specificity of PNA for nucleic acid, the PNA-DNA oligomers can be used to bind, amplify and/or detect a target nucleic acid in numerous types of samples without a significant degree of sample preparation. Thus, in one embodiment, the sample containing the target nucleic acid can be a biological sample (e.g., a carcass sample, nasal swab sample, muscous, saliva, urine, feces, whole blood, plasma, serum, cerebrospinal fluid, alveolar lavages, sweat, tears), an environmental sample (e.g., water, soil, air, sewage, food, crops, plant tissue, surface wipes and forensic sample), a contaminated sample, a suboptimal sample (e.g., bacterial culture supernatant, food sample, samples having various pH values, samples of varying salt concentrations, samples with proteins typically inhibitory to nucleic acid binding and/or a particular application, a sample with heavy metals), a purified or substantially purified sample, a pristine sample (e.g., isolated by a commercial kit (e.g., QIAGEN®) or other known procedure (e.g., maxi-prep)) or one or more combinations of any of the aforementioned samples.

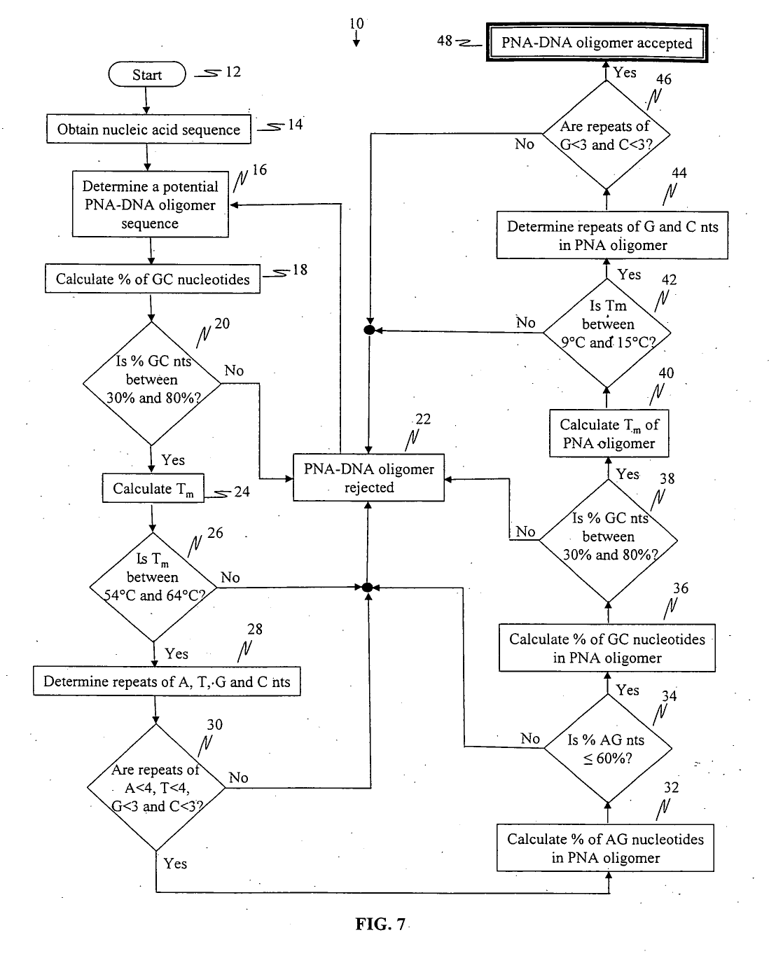

The PNA-DNA oligomer for use in the method is comprised of peptide nucleic acid bases (e.g., a PNA oligomer) and a deoxyribonucleic acids bases (e.g., a DNA oligomer) and can be designed/identified based on at least some of the constraints discussed previously (e.g., at least one PNA, limited intra-sequence complementarity, purine content of less than about 60%, GC content between about 30% to about 80%, a melting temperature of between about 54° C. and about 64° C., single nucleotide repeats of less than four A or T nucleotide bases and less than three G or C nucleotide bases). (See also, FIG. 7). In a particular embodiment, the PNA oligomer and DNA oligomer are covalently linked by a C₆ amino linker having the formula:

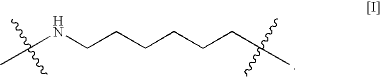

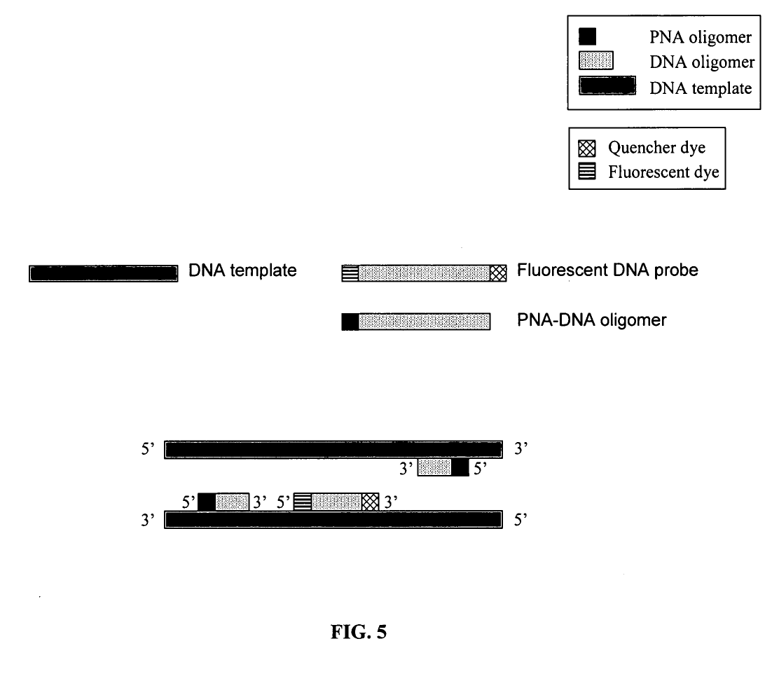

In one embodiment, the DNA polymerase for use in the method is preferably a thermostable polymerase that synthesizes DNA in the 5′ to 3′ direction and has a exonuclease activity. In a particular embodiment, the exonuclease activity of the DNA polymerase is a 3′ to 5′ exonuclease activity. Some DNA polymerases include those that are naturally-occurring like bacteriophage T7 DNA polymerase, DNA polymerase γ, E. coli DNA pol I, Thermus aquatics (Taq) pol I, Bacillus stearothermophilus (Bst) pol I, T4 DNA polymerase, Klenow DNA pol I, Vent, Pyrococcus furiosus (Pfu) DNA pol I, Thermococcus Kodakaraensis DNA polymerase as well as engineered polymerases like Phusion™ DNA polymerase (New England BioLabs) and ProofStart DNA polymerase (QIAGEN®). These DNA polymerases are well-known in the art, well-characterized and commercially available (e.g., Stratagene, Applied Biosystems, New England BioLabs, QIAGEN®). In a particular embodiment, the DNA polymerase for use in the method is Taq pol I (e.g., SureStart™ Taq, Stratagene). As DNA polymerase is only able to add a nucleotide to a 3′ hydroxyl (3′-OH) group, the PNA-DNA oligomer serves as a “primer” for the DNA polymerase and the DNA oligomer portion of the PNA-DNA oligomer provides the necessary 3′-OH. Accordingly, in this embodiment of the invention, the PNA-DNA oligomer is in a configuration in which the PNA oligomer comprises the 5′ portion of the primer and the DNA oligomer comprises the 3′ portion of the primer (see FIG. 5). This configuration allows the DNA polymerase, which does not recognize PNA, as PNA does not provide a 3′-OH group, to bind to and extend the 3′-OH group of the DNA oligomer portion of the PNA-DNA oligomer. The DNA polymerase enzymatically polymerizes/adds unlabeled deoxynucleotide triphosphates (dNTPs) to the PNA-DNA oligomer that are complementary to the target nucleic acid sequence. The dNTPs for use in the reaction are also commercially available (e.g., Fisher BioReagents, Eppendorf, TaKaRa, Promega).

The DNA polymerase extends the PNA-DNA oligomer; however, when the enzyme reaches the labeled probe, the exonuclease activity of the DNA polymerase cleaves the labeled probe in order to continue extension of the PNA-DNA oligomer. In accordance with this aspect of the method, the probe is labeled such that its degradation by the exonuclease activity of the DNA polymerase results in emission/release of a detectable signal. For example, the probe is labeled with a fluorophore (e.g., a high energy fluorescent reporter dye) on one end (e.g., the 5′ end of the probe) and a quencher moiety (e.g., a low energy quencher dye) on the other end (e.g., the 3′ end of the probe). Known as fluorescent resonance energy transfer (FRET), in this configuration, the high-energy reporter dye is in close proximity to the low energy quencher dye, resulting in a transfer of energy from the high-energy donor dye to the low energy acceptor dye that suppresses fluorescent emission of the reporter dye. Thus, when the probe is intact, the fluorophore is in close enough proximity to the quencher moiety such that emission from the fluorophore is absorbed by the quencher and no detectable fluorescent signal is emitted. To ensure that the fluorophore is in close enough proximity to the quencher moiety so that fluorescent emission of the fluorophore is suppressed, the FRET pair is generally separated by no more than 10-100 Angstroms (Å) or 1-10 nanometers (nm). Thus, for proper quenching of the fluorescent moiety in the intact probe, the probe would be, in one embodiment, no longer than about 20 to 30 nucleotides. During extension of the PNA-DNA oligomer, the DNA polymerase degrades the probe which is annealed to the target nucleic acid downstream of the PNA-DNA oligomer and releases the fluorophore from close proximity to the quencher molecule, thereby preventing FRET and allowing the fluorophore reporter to emit a detectable fluorescent signal upon excitation by a light source (e.g., halogen, laser). For FRET to take place, there should be significant overlap between the reporter dye (donor) excitation spectrum and the quencher dye (acceptor) adsorption spectrum.

In one embodiment, the fluorophore 6-carboxy-fluorescein (FAM) is covalently linked to the 5′ end of the probe and the quencher moiety 6-carboxy-tetramethylrhodamine (TAMRA) is covalently linked to the 3′ terminus of the probe. Other FRET pairs include: FAM-LC red, ROX-Cy5, FAM-Yakima Yellow™, methyl red or DABCYL-Eclipse™ Dark Quencher, BFP-GFP; CFP-dsRED; Cy3-Cy5; CFP-YFP; Alexa488-Alexa555; Alexa488-Cy3; and FITC-TRITC. Nucleic acids (e.g., probes) coupled to FRET pairs can also be obtained commercially (e.g., Molecular Probes, Intergen, Epock Biosciences, Eurogentec). Excitation of and emission by the fluorescent moiety is accomplished by exposure to a light source (e.g., halogen, laser). Irradiation of the sample and capture of the fluorescent emission can be done in the device in which the method is performed (e.g., a thermal cycler) or in a separate device either during or after the reaction.

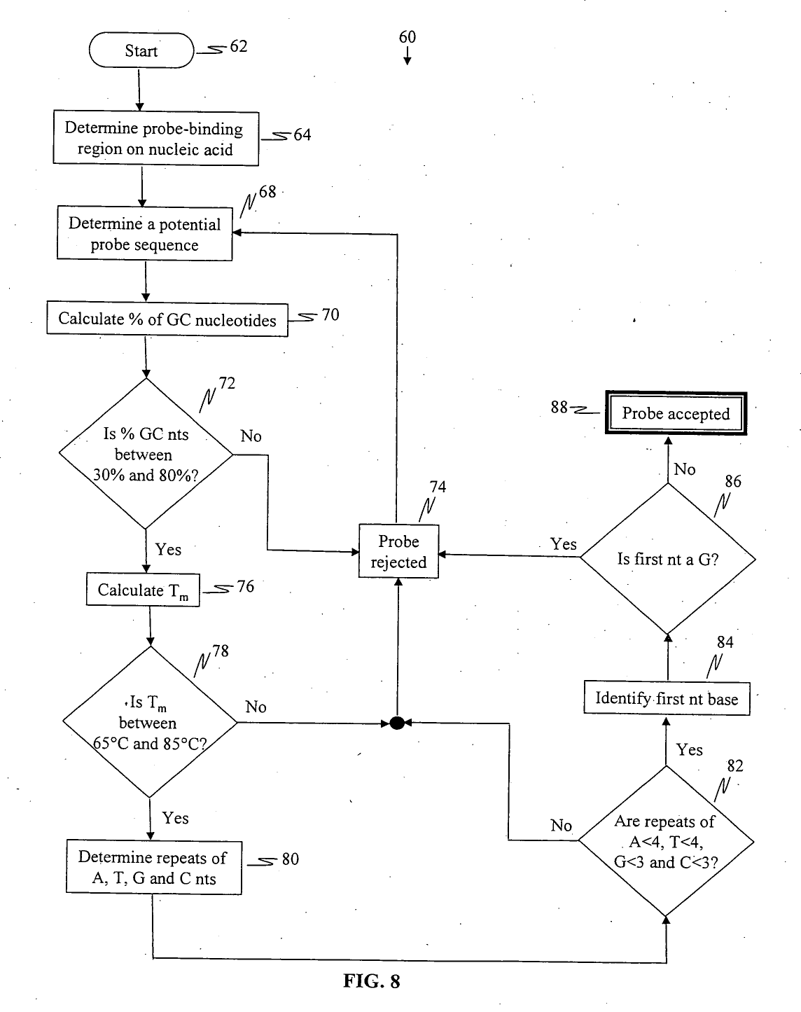

The labeled probe can be comprised of any nucleic acid or nucleic acid analog including DNA, RNA, PNA or some combination of the foregoing. The labeled probe is of a length that allows for the appropriate hybridization of the probe to the target nucleic acid and sufficient quenching of the fluorophore in the intact probe. The length of the probe is generally about 20 to about 30 nucleotide bases long and, in one embodiment, is at least about 17 nucleotide bases long. For optimal binding to the target nucleic acid, in one embodiment, the sequence of the probe would be constrained by parameters similar to those of consideration in the identification of the PNA-DNA oligomers including melting temperature, GC content, nucleotide repeats, sequence intra-complementarity, nucleic acid geometry and, when applicable (e.g., the probe is comprised, in part, of PNA), purine content (see also FIG. 8). In order for the labeled probe to be degraded by procession of the DNA polymerase, the labeled probe needs to stably bind to the target nucleic acid before the DNA polymerase begins to extend the PNA-DNA oligomer. In one embodiment, the probe is comprised of DNA whereas in another embodiment, the probe is comprised of PNA and DNA. In this embodiment, the PNA would not comprise the 5′ portion of the probe, as the DNA polymerase/exonuclease would not recognize the PNA base(s) as a substrate and degrade it to release the label. Thus, the PNA base(s) would preferably be located at, or very near, the 3′ terminus of the probe or, alternatively, in the center of the probe, flanked by DNA (e.g., a DNA-PNA-DNA oligomer).

The labeled probe binds/hybridizes to a region on the target nucleic acid downstream (e.g., 3′) of the region to which the PNA-DNA oligomer hybridizes. That is, the PNA-DNA oligomer is upstream (e.g., 5′) of the labeled probe (see FIG. 5). In a particular embodiment, the labeled probe binds to a region of the target nucleic acid sequence that is at least 1 nucleotide, 2 nucleotides, 3 nucleotides, 4 nucleotides, 5 nucleotides, 6 nucleotides, 7 nucleotides, 8 nucleotides, 9 nucleotides or 10 nucleotides downstream (e.g., 3′) of the 3′ end of the PNA-DNA oligomer to allow space for both the probe and the oligomer to bind. In a particular embodiment, the labeled probe binds to a region of the target nucleic acid sequence no more than about 5 nucleotide base pairs downstream of the 3′ end of the PNA-DNA oligomer to ensure adequate hydrolysis of the probe by the exonuclease of the DNA polymerase. After degradation of the probe, the DNA polymerase continues to extend the PNA-DNA oligomer with unlabeled dNTPs provided in the reaction, such that a full-length unlabeled target nucleic acid product is formed.

The PNA-DNA oligomer, labeled probe, dNTPs and DNA polymerase are combined with the sample, and the PNA-DNA oligomer and the labeled probe anneal (bind, hybridize) to the target nucleic acid. The PNA-DNA oligomer and labeled probe anneal to the nucleic acid under conditions that include the appropriate buffer and/or counterions (e.g., magnesium chloride (MgCl₂)) as determined by the skilled artisan for a particular sample. However, as binding of the PNA-DNA oligomer is relatively insensitive to ionic concentration and the presence of proteins inhibitory to conventional PCR, hybridization of the PNA-DNA oligomer is mostly dependent on the temperature of the combination for the particular steps of the method. In one embodiment, the PNA-DNA oligomer is annealed at temperatures between about 44° C. and about 55° C. to facilitate sequence-specific binding and, in a particular embodiment, is annealed at about 55° C. To prevent precipitation of the PNA-DNA oligomer bound to the target nucleic acid, the complex would preferably be maintained in the presence of counterions.

After hybridization, the PNA-DNA oligomer is extended under conditions suitable for extension of the PNA-DNA oligomer by the DNA polymerase using unlabeled dNTPs. Efficient extension of the PNA-DNA oligomer is dependent on the temperature and pH at which the DNA polymerase (e.g., Taq) is most effective, and this temperature can vary from between about 65° C. to about 75° C. In one embodiment, the temperature for extension of the PNA-DNA oligomer is about 72° C. Effective binding and extension of the PNA-DNA oligomer is also dependent on the use of the appropriate concentration of the PNA-DNA oligomer. Like the annealing and extension temperatures, the amount (e.g., concentration) of PNA-DNA oligomer used in the method is determined based on the particular oligomer and target nucleic acid, and can range from about 300 to about 3000 nanomolar (nM). In consideration of expense, preferably, the lowest effective amount of the PNA-DNA oligomer is used in the method and, in a particular embodiment, the PNA-DNA oligomer is added to the combination at a concentration of about 300 nM. The reaction conditions should also be suitable for the labeled probe to stably hybridize to the target nucleic acid. Specifically, in one embodiment, the labeled probe should have a high melting temperature in order to remain stably bound to the target nucleic acid upon the increase in temperature of the combination necessary for DNA polymerase extension of the PNA-DNA oligomer. Generally, the melting temperature of the probe is at least about 10° C. higher than the melting temperature of the primer (e.g., the PNA-DNA oliogmer). Due to the increased binding affinity of PNA for nucleic acid, design of a probe with a sufficiently high melting temperature can be more easily achieved with a probe containing both PNA and DNA. One of skill in the art can ably determine the appropriate annealing and extension temperatures for the identified PNA-DNA oligomer, labeled probe and DNA polymerase for use in the methods of the invention and methods of doing so are well-known in the art (see, e.g., Maniatis et al., Molecular Cloning: A Laboratory Manual, Cold Spring Harbor Laboratory, New York, 1982 and Sambrook J. et al., Molecular Cloning: A Laboratory Manual, Cold Spring Harbor Laboratory, New York, 1989).

To determine if a target nucleic acid is present, the combination is analyzed for emission of a detectable signal, which indicates hybridization of the PNA-DNA oligomer to the target nucleic acid sequence and subsequent degradation of the labeled probe due to extension of the bound PNA-DNA oligomer. In one embodiment, the detection of an emission by the fluorophore indicates the presence of the target nucleic acid in the sample. As discussed previously, through excitation by a light source (e.g., halogen, laser), this fluorescent emission can be detected and the level of fluorescence measured/quantitated in an automated system (e.g., Applied Biosystems Prism 5700, 7000, 7700 or 9700, ICycler™, Engine Opticon™, Corbett Rotorgene™, LightCycler™, MX4000™, SmartCycler™). Preferably, this detection and measurement is done in real time, e.g., while the reaction is taking place. Notably, this method does not produce a full-length labeled nucleic acid product, but instead produces an unlabeled full-length nucleic acid product. It is the probe that is labeled and this label detected upon degradation of the probe by the exonuclease activity of the DNA polymerase. In a particular embodiment, the method further comprises amplifying the full-length target nucleic acid product by methods known to those of skill in the art (e.g., polymerase chain reaction). For example, the unlabeled target nucleic acid product could be amplified by denaturing the unlabeled target nucleic acid product (at, e.g., about 95° C.), thereby producing single-stranded nucleic acid, annealing the PNA-DNA oligomer, the labeled probe and a reverse primer complementary to the strand not bound by the PNA-DNA oligomer to the denatured unlabeled target nucleic acid product, extending the PNA-DNA oligomer and reverse complementary primer, thereby degrading the labeled probe and producing additional unlabeled target nucleic acid product amplicons, and then repeating these steps (denaturation, annealing, extension, probe degradation) or, cycles, several times. The amplified target nucleic acid products are detected in real time through degradation of the labeled probe bound to the target nucleic acid product amplicons. The amplification process can be performed in a thermal cycler, which is available from several sources (e.g., ABI, Eppendorf, Fisher Scientific, Promega, MedProbe). In a particular embodiment in which the method is performed in the field, the thermal cycler is a portable one (e.g., RAZOR®, Idaho Technologies). This exponential amplification of the unlabeled target nucleic acid product exponentially increases the amount of fluorescent signal emitted, thereby enhancing signal detection, in real time. Assessment and/or quantitation of the actual levels of the target nucleic acid in a sample based on the fluorescent signal is generally done by software of the thermal cycler, and can be compared to that of a suitable control (e.g., a “housekeeping” gene with constant expression during experimental conditions), which can be identified by the skilled artisan for the particular experimental conditions and/or sample type.

Accordingly, using the method for detecting a target nucleic acid, one can detect the presence of one or more microorganisms (e.g., bacteria, viruses, protozoa, fungi), wherein, in one embodiment, emission of the detectable signal indicates the presence of a target nucleic acid characteristic of one or more microorganisms and, hence, the presence of the microorganism(s). Use of the PNA-DNA oligomers in the method would be especially advantageous in the detection of food-borne pathogens (e.g., Shiga-toxin-producing Escherichia coli (STEC)), antibiotic resistant microorganisms found, for example, in healthcare settings (e.g., Escherichia coli, staphylococcus, streptococcus) or bioterrorism agents (e.g., Bacillus anthracis) that might be present, for example, in environmental settings/samples. The microorganism(s) to be detected can be pathogenic or non-pathogenic. For example, bacteria that can be detected by the methods of the invention include, but are not limited to staphylococcus (e.g., Staphylococcus aureus (e.g., methicillin-resistant S. aureus (MRSA)), Staphylococcus epidermidis, or Staphylococcus saprophyticus), streptococcus (e.g., Streptococcus pyogenes, Streptococcus pneumoniae, or Streptococcus agalactiae), enterococcus (e.g., Enterococcus faecalis, or Enterococcus faecium), corynebacteria species (e.g., Corynebacterium diptheriae), bacillus (e.g., Bacillus anthracis), listeria (e.g., Listeria monocytogenes), Clostridium species (e.g., Clostridium perfringens, Clostridium tetanus, Clostridium botulinum, Clostridium difficile), Neisseria species (e.g., Neisseria meningitidis, or Neisseria gonorrhoeae), E. coli, Shigella species, Salmonella species, Yersinia species (for example, Yersinia pestis, Yersinia pseudotuberculosis, or Yersinia enterocolitica), Vibrio cholerae, Campylobacter species (e.g., Campylobacter jejuni or Campylobacter fetus), Helicobacter pylori, pseudomonas (e.g., Pseudomonas aeruginosa or Pseudomonas mallei), Haemophilus influenzae, Bordetella pertussis, Mycoplasma pneumoniae, Ureaplasma urealyticum, Legionella pneumophila, Treponema pallidum, Leptospira interrogans, Borrelia burgdorferi, mycobacteria (e.g., Mycobacterium tuberculosis), Mycobacterium leprae, Actinomyces species, Nocardia species, chlamydia (for example, Chlamydia psittaci, Chlamydia trachomatis, or Chlamydia pneumoniae), Rickettsia (for example, Rickettsia ricketsii, Rickettsia prowazekii or Rickettsia akari), brucella (e.g., Brucella abortus, Brucella melitensis, or Brucella suis), Proteus mirabilis, Serratia marcescens, Enterobacter clocae, Acetinobacter anitratus, Klebsiella pneumoniae and Francisella tularensis. In particular, the bacteria detected is staphylococcus, streptococcus, enterococcus, bacillus, Clostridium species, E. coli, yersinia, pseudomonas, Proteus mirabilis, Serratia marcescens, Enterobacter clocae, Acetinobacter anitratus, Klebsiella pneumoniae or Mycobacterium leprae.

Viruses that can be detected using the PNA-DNA oligomers include, but are not limited to, for example Picornavirdae (e.g., Enteroviruses (e.g., Human poliovirus 1, coxsackie B, Rhinoviruses (e.g., Human rhinovirus 1A), Hepatoviruses (e.g., Human hepatitis A virus), Cardioviruses (e.g., Encephalomyocarditis virus), Aphthoviruses (e.g., Foot-and-mouth disease virus 0)); Aviadenovirdae (e.g., Human, fowl, or porcine adenoviruses); Retroviridae (e.g., Gammaretroviruses (e.g., Mouse mammary tumor virus), Episilonretroviruses (e.g., Viper retrovirus, Walleye dermal sarcoma virus, Reticuloendotheliosis virus), Alpharetroviruses (e.g., Avian leukosis virus; Betaretrovirus: Mason-Pfizer monkey virus, Deltaretroviruses (e.g., Bovine leukemia virus, human T-lymphotropic virus (HTLV)), Lentiviruses (e.g., bovine immunodeficiency virus, Equine infectious anemia virus, feline immunodeficiency virus, caprine arthritis encephalitis virus, visna/maedi virus, human immunodeficiency virus 1, human immunodeficiency virus 2, simian immunodeficiency virus), Spumaviruses (e.g., Chimpanzee foamy virus, Human spumavirus)); Bunyaviridae (e.g., Bunyaviruses (e.g., California encephalitis virus, La Cross virus), Hantaviruses (e.g., Hantaan virus, Sin Nombre virus), Nairoviruses (e.g., Crimean-congo hemorrhagic fever virus), Phleboviruses (e.g., Sandfly fever Sicilian virus, Rift valley fever virus), Tospoviruses (e.g., Tomato spotted wilt virus)); Papovaviridae (e.g., Human papillomavirus, murine polyomavirus, Rabbit (Shope) Papillomavirus); Herpesviridae (e.g., Human Herpes Virus; Alphaherpesviruses (e.g., Simplexvirus:Herpes Simplex Virus, Human herpesvirus), Varicelloviruses (e.g., Human herpesvirus 3 (varicella-zoster virus 1), Equine Herpesvirus-1); Betaherpesviruses (e.g., Cytomegalovirus: Human Cytomegalovirus (HCMV, Human Herpesvirus 5)), Muromegaloviruses (e.g., Mouse cytomegalovirus 1), Roseoloviruses (e.g., Human Herpesvirus 6); Gammaherpesviruses, Lymphocryptoviruses (e.g., Human herpesvirus 4), Rhadinoviruses (e.g., Ateline herpesvirus 2)); Reoviridae (e.g., Colorado tick fever virus); Poxviruses (e.g., vaccinia virus); Paramyxoviridae (e.g., parainfluenza viruses 1-3); Flaviviridae (e.g., Yellow fever virus group, Tick-borne encephalitis virus group, Japanese encephalitis Group, Dengue Group); Togaviridae (e.g., Eastern/Western encephalitis viruses, Rubella virus); Filoviridae (e.g., Ebola virus, Marburg virus; Orthomyxoviridae (e.g., Influenza A virus, Influenza B virus, Influenza C virus, Togoto virus); Rhabdoviridae (e.g., Rhabdovirus, vesicular stomatitis virus, rabies virus, bovine ephemeral fever virus, lettuce necrotic yellows virus, potato yellow dwarf virus); Coronaviridae (e.g., Human Coronavirus—SARS, Avian infectious bronchitis virus, Bovine coronavirus, Canine coronavirus, Feline infectious peritonitis virus, Murine hepatitis virus, Porcine hemagglutinating encephalomyelitis virus); Parvoviridae (e.g., Parvovirus B19); Arenaviridae (e.g., Lymphocytic choriomeningitis virus) and Caliciviridae (e.g., Norwalk virus, Calicivirus).

PNA also binds robustly to ribonucleic acid (RNA). Thus, a particular target RNA (e.g., viral RNA, mRNA, ribosomal RNA) can be detected using the PNA-DNA oligomers of the invention. Accordingly, the present invention also relates to methods of detecting the presence of a target ribonucleic acid (RNA) in a sample, one such method comprising combining a first peptide nucleic acid (PNA)-DNA oligomer, a reverse transcriptase enzyme, unlabeled deoxyribonucleotide phosphates (dNTPs) and the sample, thereby forming a first combination. As before the components of the combination can be added sequentially or simultaneously, dependent, for example, on what is most appropriate for the sample conditions. The combination is maintained under conditions suitable for annealing the first PNA-DNA oligomer to the target RNA in said sample, wherein the first PNA-DNA oligomer anneals to the target RNA. The first PNA-DNA oligomer is extended with the reverse transcriptase enzyme in the presence of the unlabeled dNTPs, thereby forming an unlabeled cDNA product. The second PNA-DNA oligomer is combined with a labeled probe, a DNA polymerase having exonuclease activity and the unlabeled cDNA product, thereby forming a second combination and the second combination maintained under conditions suitable for annealing the second PNA-DNA oligomer and the labeled probe to the unlabeled cDNA product, wherein the second PNA-DNA oligomer and the labeled probe anneal to the unlabeled cDNA product, and wherein the labeled probe anneals to the unlabeled cDNA product downstream of the second PNA-DNA oligomer. This second PNA-DNA oligomer is extended with the DNA polymerase having exonuclease activity in the presence of unlabeled dNTPs, thereby forming a full-length unlabeled DNA product, wherein the exonuclease activity of the DNA polymerase degrades the labeled probe annealed to the unlabeled cDNA product during extension of the second PNA-DNA oligomer annealed to the unlabeled cDNA product, thereby resulting in emission of a detectable signal. The combination is analyzed for emission of the detectable signal, wherein emission of said detectable signal indicates the presence of the target RNA in the sample.

In accordance with this aspect of the invention, target RNA is specifically bound by a first PNA-DNA oligomer and the target RNA transcribed into DNA by the RNA-dependent DNA polymerase reverse transcriptase. This first PNA-DNA oligomer acts as a primer for the reverse transcriptase enzyme, which reverse transcribes a strand of DNA complementary to the target RNA (a cDNA). Various reverse transcriptase enzymes (e.g., murine leukemia virus (MuMLV), avian myeloblastosis (AMV), Rous-associated virus 2 (RAV-2)) are available commercially from several sources (e.g., Life Technologies, Fisher Scientific). The reverse transcriptase extends (e.g., polymerizes the addition of nucleotides to) the PNA-DNA oligomer from the 3′-OH of the DNA portion of the oligomer, adding the unlabeled dNTPs provided in the combination, to form an unlabeled complementary DNA (cDNA) product. The conditions under which the PNA-DNA oligomer anneals to the target RNA and is subsequently extended by the reverse transciptase are again, primarily dependent on the temperature of the reaction. Typically, the PNA-DNA oligomer is annealed to the target RNA at temperatures between about 50° C. and about 75° C. and the extension of the PNA-DNA oligomer performed at between about 35° C. and about 45° C., these temperatures best determined by the particular target RNA and reverse transcriptase used.

After synthesis of the unlabeled cDNA product, detection of this cDNA is then accomplished using the above-described method of detecting a target nucleic acid. Thus, the unlabeled cDNA product is combined with a second sequence specific PNA-DNA oligomer and a labeled probe forming a second combination in which, under the appropriate conditions (e.g., temperature) the second PNA-DNA oligomer and labeled probe anneal/hybridize to the target unlabeled cDNA product, the labeled probe binding the unlabeled cDNA product 3′ of the bound PNA-DNA oligomer. As before, the hybridized PNA-DNA oligomer is extended with unlabeled dNTPs by a DNA polymerase that has exonuclease activity such that, upon extension of the PNA-DNA oligomer, the exonuclease activity of the DNA polymerase degrades the probe, resulting in emission of a detectable signal. To enhance signal detection, in one embodiment, enough of the reagents of the second combination are added such that the unlabeled cDNA product is amplified, resulting in an exponential increase fluorescent emission events due to the degradation of the labeled probes by the DNA polymerase during extension of the PNA-DNA oligomers.

The second PNA-DNA oligomer, labeled probe, unlabeled dNTPs and DNA polymerase can be added to the first combination after the unlabeled cDNA product has been formed. Alternatively, a substance/composition containing the reagents for the second combination, can be added to/included in the first combination. The substance could comprise, for example, wax beads, the contents of which are released upon heating of the mixture, thereby making the second PNA-DNA oligomer and labeled probe available to hybridize to the unlabeled cDNA product.

As the PNA has a higher affinity for RNA than traditional DNA primers, in a particular embodiment, the sample in which the target RNA is detected is a biological sample, an environmental sample, a contaminated sample, a suboptimal sample, a substantially purified sample, a pristine sample and/or combinations thereof. In some cases, in the detection of, for instance, viral RNA, sample preparation is not necessary to allow the PNA-DNA oligomer to bind the RNA, as viral RNA can be found outside of the viral capsid. However, some minimal sample preparation (e.g., sonication) may be used to enable the PNA-DNA oligomer access to mRNA or ribosomal RNA, for example. In other words, PNA-DNA oligomers, increase the speed and efficiency of both the reverse transcription of target RNA into cDNA and amplification of the resultant cDNA product.

PNA-DNA oligomers can also be used to detect the presence of a target nucleic acid, in a method similar to traditional polymerase chain reaction (PCR). PCR which involves the repetition of a cycle in which DNA is denatured, specific forward and reverse primers annealed to the DNA, then extended to form DNA strands complementary to the strand bound by each primer and thus, form double-stranded DNA products (amplicons) that are subsequently amplified exponentially by repeating the process several times. However, the process of PCR is not 100% efficient. The efficiency of the reaction is largely dependent on the efficiency of primer binding to the DNA, and the percentage of molecules that are able to be extended to completion. The first PCR cycles are the least efficient, because there is the least amount of amplifiable DNA present, and because the target is mainly long (genomic, plasmid, or long amplicons). This is in contrast to later cycles, in which most of the amplification is occurring through binding to and extension of shorter amplicons. Increasing the efficiency of early PCR cycles would not only cause the reaction to reach exponential amplification faster, thereby enabling more rapid detection, but would also improve the limits of detection of a particular nucleic acid.

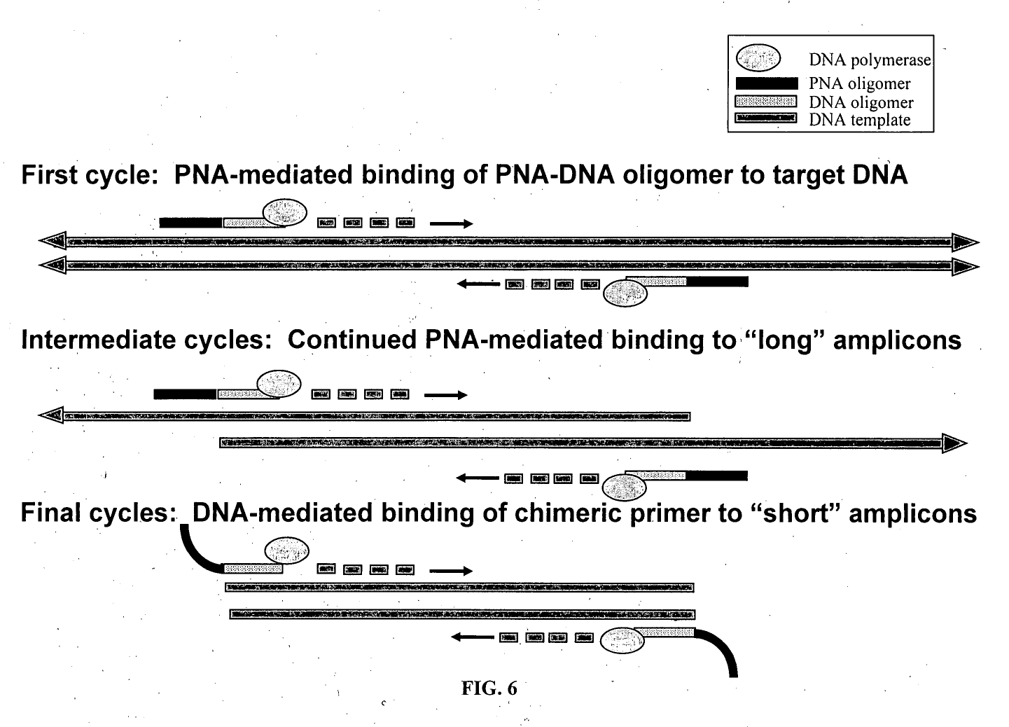

Accordingly, a method is provided for detecting the presence of a target nucleic acid that increases the efficiency of early stage PCR using the enhanced-binding ability of PNA-DNA oligomers (see FIG. 6). Thus, the method involves the hybridization of at least one PNA-DNA oligomer to a single-stranded target nucleic acid, extension of the PNA-DNA oligomer to form a full-length unlabeled double-stranded DNA products and amplification of that product(s) in subsequent cycles. Specifically, the method comprises combining, sequentially or simultaneously, a peptide nucleic acid (PNA)-DNA oligomer, DNA polymerase, unlabeled deoxyribonucleotide triphosphates (dNTPs) and the sample, thereby forming a combination. The combination is maintained under conditions suitable for annealing the PNA-DNA oligomer to the target nucleic acid in the sample, wherein the PNA-DNA oligomer anneals to the target nucleic acid. The PNA-DNA oligomer is extended with the DNA polymerase, in the presence of the unlabeled dNTPs, thereby forming a full-length unlabeled target nucleic acid product. The full-length unlabeled target nucleic acid product is amplified in a method comprising: i) denaturing the full-length unlabeled target nucleic acid product, ii) maintaining the PNA-DNA oligomer, the DNA polymerase, a reverse complementary primer and the unlabeled target nucleic acid product under conditions suitable for annealing the PNA-DNA oligomer and the reverse complementary primer to the denatured full-length unlabeled target nucleic acid product, wherein the PNA-DNA oligomer and the reverse complementary primer anneal to the denatured full-length unlabeled target nucleic acid product, iii) extending the PNA-DNA oligomer and the reverse complementary primer with the DNA polymerase, in the presence of unlabeled dNTPs, and iv) repeating steps i), ii) and iii) one or more times, thereby producing one or more full-length unlabeled target nucleic acid products. The method further comprises detecting the one or more full-length unlabeled target nucleic acid products, wherein the presence of one or more full-length target nucleic acid products indicates the presence of the target nucleic acid in the sample. The increased affinity and efficiency of the PNA-DNA oligomers allows the method to be performed in a number of sample types including biological samples, environmental samples, challenging samples and/or substantially purified samples, under various conditions.

The major components of the combination (the PNA-DNA oligomer, DNA polymerase, unlabeled dNTPs) are as detailed previously. Performance of the method (e.g., combining the components, annealing the PNA-DNA oligomer to the target nucleic acid (at, e.g., about 45° C. to about 55° C.) extending the target nucleic acid (at, e.g., about 65° C. to about 72° C.) and amplifying the full-length unlabeled target nucleic acid product) can be done in traditional thermal cyclers or in a thermal cycler that is portable for use outside the laboratory (e.g., RAZOR®). As in traditional PCR, the initial extension product (i.e., the full-length unlabeled target nucleic acid) formed in this case by extension of the PNA-DNA oligomer hybridized to the target nucleic acid, is amplified under conditions suitable for an PNA-DNA oligomer identified. Thus, the full-length unlabeled nucleic acid product can be denatured at a temperature dictated by the melting temperature of the extension product. This melting temperature can be determined by one having skill in the art using methods outlined in Maniatis et al., Molecular Cloning: A Laboratory Manual, Cold Spring Harbor Laboratory, New York, 1982 and/or Sambrook J. et al., Molecular Cloning: A Laboratory Manual, Cold Spring Harbor Laboratory, New York, 1989, for example, and can generally range from about 90° C. to about 95° C.

As in traditional PCR, a reverse complementary primer is used in the method to amplify the strand of nucleic acid complementary to that bound by the PNA-DNA oligomer, to form an identical unlabeled target nucleic acid product (amplicon). It is the dual amplification of both strands of the denatured nucleic acid that results in the exponential amplification of the unlabeled target nucleic acid product. The reverse complementary primer can be identified by methods known in the art and is again optimized for binding affinity, the appropriate melting temperature, decreased complementarity, low GC content, and limited nucleotide repeats. The reverse complementary primer can be comprised of any nucleic acid (e.g., DNA, RNA, PNA or combinations thereof) able to hybridize to the target nucleic acid and, thus, can also be a PNA-DNA oligomer. Conditions suitable for annealing the PNA-DNA oligomer and the reverse complementary primer to the denatured unlabeled target nucleic acid product are preferably the same and/or similar and would generally be similar to those conditions for the initial hybridization of the PNA-DNA oligomer to the target nucleic acid.

The PNA-DNA oligomer and reverse complementary primer, bound to their respective strands of nucleic acid, are extended under the appropriate conditions (e.g., temperature), by the DNA polymerase in the presence of unlabeled dNTPs for the production of full-length unlabeled target nucleic acid products. The amplification process of the method (target nucleic acid product denaturation, oligomer/primer binding and target nucleic acid product strand synthesis) are repeated one or more times (e.g., in one or more cycles) and, in most cases, numerous times, to amplify the unlabeled target nucleic acid products. The number of cycles used to amplify the unlabeled target nucleic acid products is dependent on the initial amount/level of target nucleic acid and the efficiency of primer (e.g., PNA-DNA oligomer) hybridization and extension, with the number of amplification cycles typically ranging from about 10 to about 100 cycles, about 25 to about 75 cycles, about 30 to about 45 cycles or about 40 to about 150 cycles. Generally, the determination of the optimal number of amplification cycles would primarily be based on the number of cycles deemed necessary to detect the nucleic acid extension products (e.g., the one or more full-length unlabeled target nucleic acid products). This determination is also well-within the capabilities of one of skill in the art and is known in the art.

The one or more unlabeled target nucleic acid products can be detected in a number of ways either during or after completion of the method. For example, the target nucleic acid products can subjected to an electrophoretic (e.g., agarose or acrylamide gel) or chromatographic (e.g., a column matrix) process that separates the contents of the reaction by size and/or charge. For instance, the target nucleic acid products can be visualized by exposing a gel to a dye or agent that binds nucleic acid and exciting the dye/agent with an appropriate light source. Such agents include those that intercalate between DNA (e.g., ethidium bromide, SYBR Green I™, DiSc₂(5)). The target nucleic acid products can also be detected and/or quantified during the reaction in real time using the intercalating dye SYBR Green I™ (Molecular Probes), which emits at 520 nanometers (nm). Thus, as target nucleic acid product accumulates, the fluorescent signal of SYBR Green I™ would increase. Alternatively, several other types of probes exist that could be used to detect the unlabeled target nucleic acid product, during and/or after amplification including Molecular Beacons (original and wave-shifting) (Molecular Probes), Scorpions™ or Duplex Scorpions™ (Molecular Probes), which encompass both the primer and probe function (e.g., PNA-DNA oligomer sequence and extension product binding sequence), and hybridization probes (Roche), all of which depend on FRET to generate a detectable fluorescent signal. Hybridization probes are comprised of two adjacently-binding probes, one with a 3′ donor label (e.g., FAM, ROX) and the other with a 5′ acceptor dye (e.g., LC red, Cy5), where, upon excitation, the 3′ label passes its energy to the 5′ dye through FRET and fluorescence of the dye is detected. Molecular Beacon and Scorpion™ probes rely on the formation of stem-loop structure to keep the fluorescent moiety quenched; upon hybridization to target nucleic acid (Molecular Beacons) and/or extended amplicon (Scorpions™), the fluorophore and quencher are separated and a detectable signal emitted when the fluorescent dye is irradiated by a light source. Other probes include minor groove binders (e.g., Hoechst, MGB1, TaqMan® (Applied Biosystems)), ResonSense probes, light-up probes and Hy-Beacon probes, which also use FRET to report binding. Hybridization-specific probes, generally provide additional specificity to the assay and, therefore, increased reliability with respect to identifying a particular target nucleic acid in a sample. In addition, labeled probes allow multiple DNA species to be detected/measured in the same sample. Thus, the method of the invention can be used to detect one or more target nucleic acids, through the use of one or more PNA-DNA oligomers and labeled probes, differentiation between each target nucleic acid afforded by the labeling of each specific probe with fluorophore moieties that fluoresce in different color spectrums upon excitation by light.

A method is also provided for amplifying a target nucleic acid such that target nucleic acid can be detected during this amplification. The method comprises combining a peptide nucleic acid (PNA)-DNA oligomer, a reverse complementary primer, a labeled probe, a DNA polymerase having exonuclease activity, unlabeled deoxyribonucleotide triphosphates (dNTPs) and the target nucleic acid, thereby forming a combination. The PNA-DNA oligomer, reverse complementary primer, labeled probe and DNA polymerase can be combined simultaneously and/or the components can be added sequentially. In one embodiment, the target nucleic acid is denatured. The combination is maintained under conditions suitable for extending said PNA-DNA oligomer in the presence of the target nucleic acid. The PNA-DNA oligomer, reverse complementary primer and labeled probed are annealed to the target nucleic acid, wherein the labeled probe anneals to the target nucleic acid downstream of the PNA-DNA oligomer and, under conditions suitable for extension of the PNA-DNA oligomer, the DNA polymerase extends the PNA-DNA oligomer in the presence of the unlabeled dNTPs, thereby forming full-length unlabeled target nucleic acid product. The exonuclease activity of the DNA polymerase degrades the labeled probe annealed to the target nucleic acid during extension of the PNA-DNA oligomer annealed to the target nucleic acid, which results in the emission of a detectable signal. The full-length unlabeled target nucleic acid product formed is denatured. The above steps (i.e., PNA-DNA oligomer and reverse complementary primer annealing and extension to form full-length unlabeled target nucleic acid product and denaturation of the full-length unlabeled target nucleic acid product) are repeated one or more times, thereby forming one or more unlabeled target nucleic acid products thereby amplifying the target nucleic acid. As described previously, amplification of the target nucleic acid and emission of the signal can be detected in real time through degradation of the labeled probe (m, e.g., FRET configuration).

In another method of amplifying a target nucleic acid, the present invention provides for a method that comprises combining sequentially or simultaneously a peptide nucleic acid (PNA)-DNA oligomer, a reverse complementary primer, a DNA polymerase, unlabeled deoxyribonucleotide triphosphates (dNTPs) and the target nucleic acid, thereby forming a combination. The combination is maintained under conditions suitable for annealing the PNA-DNA oligomer and the reverse complementary primer to the denatured target nucleic acid, wherein the PNA-DNA oligomer and the reverse complementary primer anneal to the target nucleic acid. The PNA-DNA oligomer and the reverse complementary primer are extended with the DNA polymerase in the presence of unlabeled dNTPs, thereby forming full-length unlabeled target nucleic acid product and the full-length unlabeled target nucleic acid product denatured. Then PNA-DNA oligomer and reverse complementary primer binding and extension and full-length unlabeled target nucleic acid product denaturation is repeated one or more times, thereby forming one or more full-length unlabeled target nucleic acid products and amplifying the target nucleic acid. The amplified full-length, unlabeled target nucleic acid products can be detected in real-time or after amplification in a number of ways, including using target nucleic acid sequence specific probes (e.g., hybridization probes, Molecular Beacons, Scorpion™) or DNA intercalators (e.g., SYBR Green I™, ethidium bromide) upon excitation by the light source of a thermal cycler.

The PNA-DNA oligomer is as described previously and, in a particular embodiment, is comprised of a PNA oligomer and a DNA oligomer that are covalently linked by a C₆ amino linker having the formula:

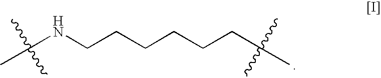

In addition, the reverse complementary primer that binds to and extends the nucleic acid strand complementary to the one bound by the PNA-DNA oligomer can be comprised of nucleic acid (e.g., DNA) or a combination of nucleic acid and nucleic acid analogs (e.g., DNA and PNA). In one embodiment, the reverse complementary primer is comprised of a PNA oligomer and DNA oligomer, that forms a second PNA-DNA oligomer.

The PNA-DNA oligomers of the invention can also be used in various other applications in which short, synthetic oligonucleotides are employed (e.g., to probe nucleic acid, sequence DNA and/or detect mutations). For instance, a method of probing a target nucleic acid sequence is provided, the method comprising hybridizing to the target nucleic acid an unlabeled peptide nucleic acid (PNA)-DNA oligomer comprising at least one PNA oligomer and at least one DNA oligomer, wherein the at least one PNA oligomer and at least one DNA oligomer are covalently linked by a linker selected from the group consisting of a C₆ amino linker having the formula:

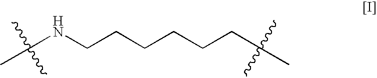

and a C5 carboxy linker having the formula:

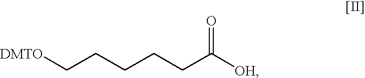

where DMTO is 3,5-dimethyl-1,2,4-trioxolane, and detecting the hybridized unlabeled PNA-DNA oligomer.

The PNA-DNA oligomer probe can be comprised of any number of PNA nucleotide bases (e.g., at least one PNA) and DNA nucleotide bases (e.g., at least one DNA) adequate for binding of the oligomer to specific sequence of the target nucleic acid. For example, for greater binding affinity and stability to the target nucleic acid, the PNA-DNA oligomer could be comprised primarily of PNA nucleotide bases. The PNA oligomer and DNA oligomer can be linked by the C₆ amino linker with the PNA at the 5′ portion of the oligomer and the DNA at the 3′ portion of the oligomer (e.g., PNA-linker-DNA). In an alternative embodiment, the DNA can be at the 5′ portion of the oligomer linked to the PNA at the 3′ portion of the oligomer, using, for example, the C₅ carboxy linker (e.g., DNA-linker-PNA). In yet another embodiment, stretches of PNA and DNA could be linked in an alternating manner using the C₆ amino and/or the C₅ carboxy linker (e.g., a PNA-DNA-PNA oligomer). The number of PNA and DNA nucleotide bases, the linkage of the PNA oligomer and DNA oligomer and the number of PNA and DNA oligomers used, is best determined by the skilled artisan based on the particular target nucleic acid and concomitant sequence being probed. Again, the target nucleic acid probed by the PNA-DNA oligomer could be any selected from the group consisting of synthetic DNA, genetically engineered DNA, genomic DNA, chromosomal DNA, mitochondrial DNA, transposon DNA, plasmid DNA, viral DNA, synthetic RNA, RNA, ribosomal RNA and viral RNA. The probe can also comprise a PNA oligomer and a RNA oligomer, forming a PNA-RNA oligomer to further enhance binding of the probe to RNA, for instance. An RNA oligomer can be linked to a PNA oligomer in a manner similar to that described above for linkage of a DNA oligomer to a PNA oligomer.

Binding of the unlabeled PNA-DNA oligomer to the target nucleic acid sequence can be detected in numerous ways. For instance, in a particular embodiment, the hybridized unlabeled PNA-DNA oligomer is detected by the cyanine dye 3,3′-diethylthiadicarbocyanine iodide (DiSc₂(5)). The DiSc₂(5) dye only binds PNA in complex with DNA and, upon binding to PNA-DNA complexes, changes in color from blue to purple. This change in color is detectable by visible light and can be measured (e.g., in a spectrophotometer). The unlabeled PNA-DNA oligomer probe could also be detected by binding of another labeled DNA probe (e.g., a fluorescently labeled probe, Molecular Beacon, hybridization probes) to the PNA-DNA oligomer probe. In addition, the hybridized PNA-DNA oligomer could be detected by an antibody that specifically binds the PNA portion of the PNA-DNA oligomer probe, the antibody labeled (e.g., by fluorescent label, biotin, gold particle) or the antibody subsequently bound by another antibody that is labeled.

Further, with enhanced nucleic acid binding ability, the PNA-DNA oligomers are useful in a number of other applications in which primers or probes are typically used. For instance, the PNA-DNA oligomers of the invention can also be used in a method of determining the sequence of a specific nucleic acid or particular region of nucleic acid by known methods (see e.g., Sanger, F et al., PNAS 74:5463-5467, 1977; Smith, L M et al., Nucleic Acids Research, 13:2399-2412, 1985; Smith, L M et al., Nature, 321:674-679, 1986; Dovichi, N J, Electrophoresis 18:2393-2399, 1997). Generally the method comprises combining an unlabeled PNA-DNA oligomer, DNA polymerase and a sample containing the nucleic acid of interest in conditions under which the PNA-DNA oligomer hybridizes to the target nucleic acid and then, in conditions under which the PNA-DNA oligomer is extended by the DNA polymerase. The extension of the PNA-DNA oligomer can be in the presence of unlabeled terminating nucleotides (e.g., nucleotides that do not allow continued DNA elongation) 2′,3′-dideoxynucleotide 5′-triphosphates (ddNTPs) or the like. This would generate a series of differentially-sized extension products, terminated at a particular nucleotide base (e.g., A, T, G or C). The terminated extension products can then resolved/separated by size using high-resolution non-denaturing electophoresis (e.g., polyacrylamide gel) or chromatography (e.g., column matrix) and visualized by exposure to a dye like SYBR Green I™ or DiSc₂(5), for example. Automated sequencing systems available include slab gel sequencing devices (Li-Cor) and capillary systems (Applied Biosystems).

The PNA-DNA oligomers can also be used to detect single nucleotide polymorphisms (SNPs) by direct sequencing of the region of interest using the PNA-DNA oligomers as described above. Alternatively, SNPs in a target nucleic acid could be detected based on mismatched binding between the PNA-DNA oligomer and the target nucleic acid. Thus, a PNA-DNA oligomer can be designed to have a sequence complementary to the wildtype DNA sequence of interest. In one instance, a mismatch between the PNA-DNA oligomer and the target nucleic acid due to a mutation in the target nucleic acid can be detected by the presence or absence of amplicons in a PNA-DNA oligomer extension reaction. The PNA-DNA oligomer complementary to the wildtype sequence can be combined with a DNA polymerase and a sample containing the nucleic acid of interest. The combination is then maintained under conditions that allow the PNA-DNA oligomer to hybridize to the target nucleic acid and then be extended by the DNA polymerase in the presence of unlabeled dNTPs, thereby forming an unlabeled target nucleic acid product. However, amplification (and/or extension) of the target nucleic acid product using the PNA-DNA oligomer (and a reverse complementary primer), would be inhibited/prevented by just a single nucleotide base pair mismatch between the PNA-DNA oligomer and the nucleic acid sequence to which it is bound. Consequently, the target nucleic acid product would not be amplified and no target nucleic acid products would be detected. Thus, the presence of a SNP would be indicated by a lack of target nucleic acid product, as assayed during or after the reaction (using e.g., a dye or labeled probe). Moreover, the presence of a mutation in a nucleic acid sequence can be demonstrated and/or confirmed through a melt curve analysis. Mismatch between the PNA portion of the PNA-DNA oligomer and target nucleic acid due to mutation in the sequence of the target nucleic acid would cause the oligomer to dissociate from the target nucleic acid at a significantly decreased temperature (e.g., a decrease in melting temperature of about 15° C. on average, for a PNA-DNA oligomer 15 bases long) as compared to its dissociation from a fully complementary wildtype sequence. The melting temperature of the complexes can be determined fluorescently or visually by a dye indicating DNA/DNA (e.g., SYBR Green I™) or PNA/DNA (DiSc₂(5)) complexes, or a lack thereof. Thus, the dye would no longer be detected or the color of the solution would change at the temperature at which the oligomer and nucleic acid dissociate. A decreased melting temperature of the PNA-DNA oligomer bound to the target nucleic acid in the sample would indicate the presence of a SNP.

The PNA-DNA oligomers are also useful in other applications in which oligonucleotide probes are typically used including, for example, microarrays (e.g., gene chips), in situ hybridization and DNA mutation detection (e.g., detection of deletions, additions, insertions, amplifications). Further, the PNA-DNA oligomers can be used as antisense agents to inhibit the expression of a particular gene product. The binding specificity, increased binding affinity and cell membrane permeability make PNA-DNA oligomers ideal antisense molecules.

Kits containing the PNA-DNA oligomers and one or more appropriate reagents to detect a target nucleic acid, a specific sequence of a target nucleic acid or a mutation in a target nucleic are also provided herein. Thus, the one or more appropriate reagents can include buffers, dyes or fluorescent reagents, antibodies, primer extension reagents (e.g., mono- or divalent cations, detergents, dNTPs, one or more DNA polymerases) and/or sequencing reagents (e.g., cations, ddNTPs, one or more DNA polymerases). The kits can further comprise operating instructions. These kits can be used experimentally in laboratories or in the field in diagnostic devices to detect a pathogenic or otherwise harmful microorganism, for example.

Methods are also provided for designing one or more PNA-DNA oligomers and/or a probe optimized for binding to a particular nucleic acid sequence. In addition to the usual constraints involved in conventional nucleic acid probe and/or primer design such as primer length, melting temperature, GC content, intra-molecular binding and nucleotide repeats, additional factors with respect to the PNA portion of the PNA-DNA oligomer must be considered in the design of the oligomers.

Thus, a method for designing such a PNA-DNA oligomer is provided that comprises obtaining the sequence of the target nucleic acid and determining a complementary PNA-DNA oligomer sequence for a region on the target nucleic acid, thereby identifying a potential PNA-DNA oligomer. The potential PNA-DNA oligomer is then accepted or rejected in a method comprising: calculating the percent of guanine (G) and cytosine (C) nucleotides in the potential PNA-DNA oligomer, wherein if the percent of G and C nucleotides is between about 30% and about 80%, then the potential PNA-DNA oligomer is accepted. The melting temperature of the potential PNA-DNA oligomer is also calculated, wherein if the melting temperature is between about 54° C. and about 64° C., then the potential PNA-DNA oligomer is accepted. The number of contiguous adenine (A), contiguous thymine (T), contiguous guanine (G) and contiguous cytosine (C) nucleotides in the potential PNA-DNA oligomer is determined, wherein if there are less than: (a) four contiguous A nucleotides, (b) four contiguous T nucleotides, (c) three contiguous C nucleotides and (d) three contiguous G nucleotides, then the potential PNA-DNA oligomer is accepted. For the PNA oligomer portion of the potential PNA-DNA oligomer, the method further comprises calculating the percent of adenine (A) and guanine (G) nucleotides, thereby determining the purine content of the potential PNA-DNA oligomer, wherein if the percent of A and G nucleotides is less than or equal to about 60%, then the potential PNA-DNA oligomer is accepted. The percent of guanine (G) and cytosine (C) nucleotides in the PNA oligomer portion of the potential PNA-DNA oligomer is also calculated, wherein if the percent of G and C nucleotides is between about 30% and about 80%, then the potential PNA-DNA oligomer is accepted. The method also comprises calculating the melting temperature of the PNA oligomer portion of the potential PNA-DNA oligomer, wherein if the melting temperature is between about 9° C. and about 15° C., then the potential PNA-DNA oligomer is accepted. In addition, the number of contiguous G and C nucleotides in the PNA oligomer portion of the potential PNA-DNA oligomer is determined, wherein if there are not three contiguous G or three contiguous C nucleotides, then the PNA-DNA oligomer is accepted. In a particular embodiment, the method is performed by a computer.

The method can be used to design a PNA-DNA oligomer for use in various applications, particularly, as forward and/or reverse primers in PCR (e.g., traditional PCR, RT-PCR and real-time PCR). One aspect of the invention relates to a PNA-DNA oligomer designed by the above method. In a particular embodiment, the PNA-DNA oligomer designed by the method is linked by the C₆ amino linker having the formula:

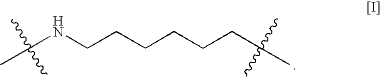

Computer program products for designing a PNA-DNA oligomer and/or are also provided. The computer program products of the invention can take the form of an entirely hardware embodiment, an entirely software embodiment or an embodiment containing both hardware and software elements. In a preferred embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate or transport the program for use by or in connection with the instruction execution system, apparatus or device.

The medium can be an electronic, magnetic, optical, electromagnetic, infrared, or semiconductor system (or apparatus or device) or a propagation medium. Examples of a computer-redable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

Input/output or I/O devices (including but not limited to keyboards, displays, pointing devices, etc.) can be coupled to the system either directly or thorough intervening I/O controllers.

Network adapters may also be coupled to the system to enable the data processing system to become coupled to other data processing systems or remote printers or storage devices through intervening private or public networks. Modems, cabel modem and Ethernet cards are just a few of the currently available types of network adapters.

Specifically, a PNA-DNA oligomer can be designed by a computer program product of the invention that comprises a computer useable medium including a computer readable program, wherein the computer readable program, when executed on a computer also causes the computer to obtain the sequence of the target nucleic acid. The computer readable program causes the computer to determine a complementary PNA-DNA oligomer sequence for a region on the target nucleic acid, thereby identifying a potential PNA-DNA oligomer. The computer readable program causes the computer to calculate the percent of guanine (G) and cytosine (C) nucleotides in the potential PNA-DNA oligomer, wherein if the percent of G and C nucleotides is between about 30% and about 80%, then the computer accepts the potential PNA-DNA oligomer. The computer readable program also causes the computer to calculate the melting temperature of the potential PNA-DNA oligomer, wherein if the melting temperature is between about 54° C. and about 64° C., then the computer accepts the potential PNA-DNA oligomer. In addition, the computer readable program causes the computer to determine the number of contiguous adenine (A), contiguous thymine (T), contiguous guanine (G) and contiguous cytosine (C) nucleotides in the potential PNA-DNA oligomer, wherein if there are less than: (a) four contiguous A nucleotides, (b) four contiguous T nucleotides, (c) three contiguous C nucleotides and (d) three contiguous G nucleotides, then the computer accepts the potential PNA-DNA oligomer. The computer readable program further causes the computer to calculate the percent of adenine (A) and guanine (G) nucleotides in the PNA oligomer portion of the potential PNA-DNA oligomer, thereby determining the purine content of the potential PNA-DNA oligomer, wherein if the percent of A and G nucleotides is less than or equal to about 60%, then the computer accepts the potential PNA-DNA oligomer. The computer readable program causes the computer to calculate the percent of guanine (G) and cytosine (C) nucleotides in the PNA oligomer portion of the potential PNA-DNA oligomer, wherein if the percent of G and C nucleotides is between about 30% and about 80%, then the computer accepts the potential PNA-DNA oligomer. Further, the computer readable program causes the computer to calculate the melting temperature of the PNA oligomer portion of the potential PNA-DNA oligomer, wherein if the melting temperature is between about 9° C. and about 15° C., then the computer accepts the potential PNA-DNA oligomer. The computer readable program, in addition, causes the computer to determine the number of contiguous G and C nucleotides in the PNA oligomer portion of the potential PNA-DNA oligomer, wherein if there are not three contiguous G or three contiguous C nucleotides, then the computer accepts the potential PNA-DNA oligomer.

For a given nucleic acid sequence, the particular constraints in the algorithm for the identification of an acceptable/optimal complementary PNA-DNA oligomer are based on a number of factors. In order to ensure specific and stable hybridization to the target nucleic acid, the PNA-DNA oligomer should have an overall GC content of at least about 30%. However, to minimize non-specific annealing of the PNA-DNA oligomer to the target nucleic acid and subsequent amplification of non-target nucleic acid (e.g., mispriming), its overall GC content should be no greater than about 80%. The ability of the PNA-DNA oligomer to hybridize to the target nucleic acid is also determined by the melting temperature (Tₘ) of the bound PNA-DNA oligomer. Based on the PNA-DNA oligomer sequence, the melting temperature can be calculated as the number of A and T nucleotides multiplied by two plus the number of G and C nucleotides multiplied by four following the equation: Tₘ=AT*2+GC*4. According to the method, the melting temperature of the PNA-DNA oligomer is acceptable if it is between about 54° C. and about 64° C. Moreover, the number of contiguous nucleotides of the same base (i.e., single nucleotide repeat) allowed in the PNA-DNA oligomer is a further design constraint. Single nucleotide repeats can lead to ambiguous binding (slippage) of oligonucleotides at their target sites and can generate secondary binding sites for the PNA-DNA oligomer. This stable hybridization of the oligomer at non-specific binding sites would reduce the efficiency of the PNA-DNA oligomer in several applications (e.g., PCR, sequencing). Accordingly, in a particular embodiment, for the A and T base pairs there are no single nucleotide repeats longer than three base pairs and for G and C base pairs that there be no more than two such bases in a row.

Most of these parameters are also specifically assessed for the PNA portion of the PNA-DNA oligomer. Thus, with respect to the PNA oligomer, the GC content should be between about 30% and about 80%, the melting temperature between about 9° C. and about 15° C., and preferably there are no G or C nucleotide repeats of 3 or more bases. As discussed previously, the PNA-oligomer should be long enough so that the PNA-DNA oligomer binds the nucleic acid with high affinity. In applications such as PCR and sequencing, the PNA portion should also to be short enough to allow the oligomer to dissociate from the template. Moreover, specific consideration should be given to the purine content of the PNA oligomer. Purine-rich PNA oligomers tend to aggregate and have low solubility in aqueous solutions. Thus, in a particular embodiment, the purine content of the PNA portion of the PNA-DNA oligomer is less than about 60%.

Depicted in FIG. 7 is a flow chart illustrating an example of processing performed by a microcontroller in design of a PNA-DNA oligomer for a nucleic acid sequence. Process 10 begins with block 14 in which the sequence of the nucleic acid of interest is obtained (e.g., nucleic acid sequence is entered). In block 16, a potential PNA-DNA oligomer sequence is determined based on sequence complementary to a region of the nucleic acid sequence. The percent of G and C nucleotides in the potential PNA-DNA oligomer sequence is calculated in block 18 and if the percent of G and C nucleotides in the oligomer is between 30% and 80%, process 10 proceeds from decision block 20 to block 24. However, if at any point in process 10 the PNA-DNA oligomer does not meet the criteria set forth in any decision block, process 10 proceeds to block 22, in which the potential PNA-DNA oligomer is rejected. Process 10 then returns to block 16, to determine a different PNA-DNA oligomer that is complementary to a region of the nucleic acid. In block 24, the melting temperature Tₘ of the potential PNA-DNA oligomer is calculated. If the melting temperature of the potential PNA-DNA oligomer is between 54° C. and 64° C., then process 10 proceed to block 28, in which the number of single nucleotide in the sequence is assessed. If, in decision block 30, it is determined that the potential PNA-DNA oligomer sequence contains less than 4 nucleotide repeats of A and T and less than 3 nucleotide repeats of G and C, then process 10 proceeds to block 32. In block 32, the percent of A and G nucleotides in the PNA oligomer portion of the potential PNA-DNA oligomer is calculated, and if in decision block 34 the percent of A and G nucleotides is found to be less than or equal to 60%, then process 10 proceeds to block 36 in which the G and C content of the PNA oligomer of the potential PNA-DNA oligomer is calculated. If the percent of G and C nucleotides is between 30 and 80% in decision block 38, then process 10 proceeds to block 40. The melting temperature of the PNA portion of the potential PNA-DNA oligomer is calculated in block 40 and if this melting temperature is found to be between 9° C. and 15° C. in decision block 42, then process 10 proceeds to block 44. In block 44 the number of G and C single nucleotide repeats in the PNA oligomer portion of the potential PNA-DNA oligomer is determined. If the G and C nucleotides are both repeated less than three times in the PNA oligomer then the potential PNA-DNA oligomer is accepted.

The method of designing a PNA-DNA oligomer can further comprise the design of a probe, specifically, one that would bind downstream of the PNA-DNA oligomer in a method of detecting a target nucleic acid. As with the design of the PNA-DNA oligomer, in a particular embodiment, the probe also has a GC content of between about 30% and about 80% and has three or less (e.g., less than four) A and T single nucleotide repeats and two or less (e.g., less than three) G and C nucleotide repeats. In addition, in another particular embodiment, the first nucleotide of the probe (e.g., the first base at the 5′ end of the probe) is not a guanine.

The probe can be of any length as deemed necessary for a particular application. In the instance that the probe is labeled, for example, in a FRET format and contains no intentional hairpins or loops (as in, e.g., Molecular Beacons or Scorpions™), the probe is generally no longer than about 20 to about 30 bases to maintain adequate quenching of the fluorophore reporter moiety by the quenching moiety. For distances of greater than 10 nm (e.g., about 30 nucleotide bases), fluorophore quenching falls off rapidly. Furthermore, in particular embodiments, a guanine (G) is not the first nucleotide at the 5′ end of such a probe (e.g., a probe labeled in a FRET format), as the G quenches the fluorophore on the 5′ end of the probe. Thus, in this embodiment, even if the probe was cleaved/degraded by a DNA polymerase in real-time PCR, for example, this cleavage event cannot be detected as the G continues to quench the fluorophore despite the fluorphore no longer being in close proximity to the quenching moiety.

The distance of the probe from the region at which the PNA-DNA oligomer binds has a significant effect on probe hydrolysis, which, in some applications (e.g., real-time PCR), is necessary to generate a detectable signal. In particular embodiments, the distance between the upstream PNA-DNA oligomer (i.e., the 3′ end of the PNA-DNA oligomer) and the interceding probe (i.e., the 5′ end of the probe) is no less than one base pair so that there is space for both the PNA-DNA oligomer and the probe to bind to target nucleic acid sequence. In some embodiments, this distance is no more than five base pairs to ensure adequate hydrolysis of the probe by the DNA polymerase. In yet another embodiment, the melting temperature of a potential probe is between about 65° C. and about 85° C. to ensure stable binding of the probe during the increase in temperature required for activity of the DNA polymerase for extension of the PNA-DNA oligomer. The melting temperature (Tₘ) of the probe sequence can be calculated in the same way as calculation of the Tₘ for the PNA-DNA oligomer, where the Tₘ=AT*2+GC*4.

In one embodiment, the method of designing a probe is performed in a computer. In addition, the present invention also provides for a computer program product that designs a probe. The computer program product comprises a computer readable medium including a computer readable program, wherein the computer readable program, when executed on a computer causes the computer to determine a probe-binding region on the target nucleic acid located between 1 and 5 nucleotides 3′ to the region that the PNA-DNA oligomer binds the target nucleic acid. Execution of the computer readable program causes the computer to also determine a complementary nucleotide sequence for the probe-binding region, thereby identifying a potential probe. The computer readable program further causes the computer to calculate the percent of cytosine (C) and guanine (G) nucleotides in the potential probe, wherein if the percent of C and G nucleotides is between about 30% and about 80%, then the computer accepts the potential probe. In addition, the computer readable program causes the computer to calculate the melting temperature for the potential probe, wherein if the melting temperature is between about 65° C. and about 85° C. then the computer accepts the potential probe. Execution of the computer readable program also causes the computer to determine the number of contiguous adenine (A), contiguous thymine (T), contiguous guanine (G) and contiguous cytosine (C) nucleotides in the potential probe, wherein if there are less than: (a) four contiguous A nucleotides, (b) four contiguous T nucleotides, (c) three contiguous C nucleotides and (d) three contiguous G nucleotides, then the computer accepts the potential probe. The computer readable program causes the computer to identify the first nucleotide base of the potential probe as well, wherein if the first nucleotide base is an adenine (A), thymine (T) or a cytosine (C), then the computer accepts the potential probe.

FIG. 8 depicts a flow chart illustrating an example of processing performed in a microcontroller for design of a probe as part of a PNA-DNA oligomer/probe set in real-time PCR, for example. Process 60 begins with block 64 in which a probe-binding region is identified on the nucleic acid. As discussed above, this probe-binding region would generally be between one and 5 nucleotide bases downstream of the bound PNA-DNA oligomer. Based on that probe-binding region, a potential probe sequence, complementary to the region is determined in block 70. The percent of G and C nucleotides in the potential probe sequence is calculated and if, in decision block 72 the percent of G and C nucleotides is between 30% and 80%, then process 60 proceeds to block 76. In block 76, the melting temperature of the probe is calculated and if the melting temperature is found to be between 65° C. and 85° C. in block 78, then process 60 proceeds to block 80 in which single nucleotide repeats of A, T, G and C in the potential probe are determined. In block 82, if the potential probe contains less than 4 nucleotide repeats of A and T and less than 3 nucleotide repeats of G and C, then process 80 proceeds to block 84. The first nucleotide base in the potential probe sequence is identified in block 28 and if this first nucleotide is not a G (e.g., is an A, T or C), then the potential probe is accepted. As with design of the PNA-DNA oligomer, if during process 60, in any of the decision blocks the criteria outlined are not met, then the probe is rejected in block 74 and process 60 returns to block 68 to identify another potential probe. If an appropriate probe can not be identified for a particular PNA-DNA oligomer in process 60, it may be necessary to return to process 10 and design a different PNA-DNA oligomer.

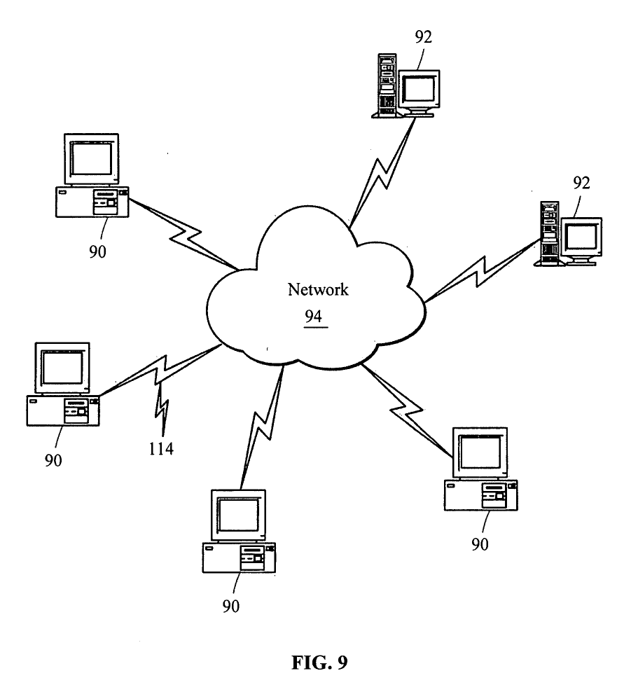

FIG. 9 illustrates a computer network or similar digital processing environment in which the present invention may be implemented. Client computer(s)/devices 90 and server computer(s) 92 provide processing, storage, and input/output executing application programs and the like. Client computer(s)/devices 90 can also be linked through communications network 94 to other computing devices, including other client devices/processes 90 and server computer(s) 92. Communications network 94 can be part of a remote access network, a global network (e.g., the Internet), a worldwide collection of computers, Local area or Wide area networks and gateways that currently use respective protocols (TCP/IP, Bluetooth, etc.) to communicate with one another. Other electronic device/computer network architectures are suitable.

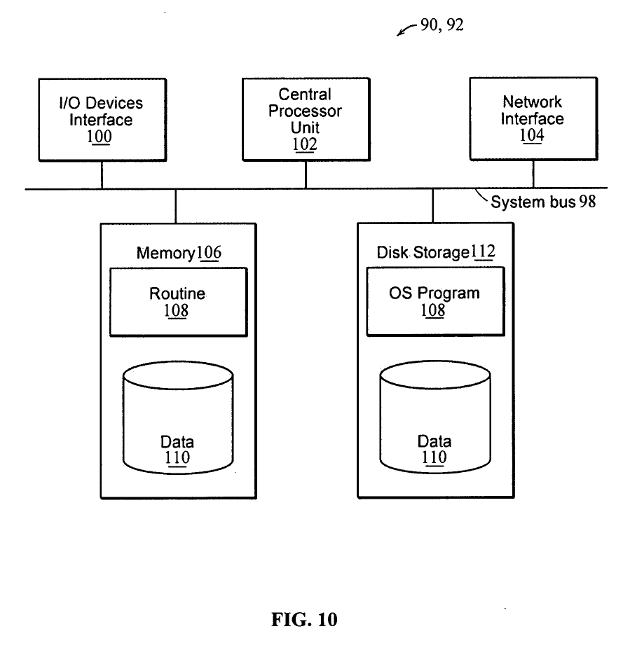

FIG. 10 is a diagram of the internal structure of a computer (e.g., client processor/device 90 or server computers 92) in the computer system of FIG. 9). Each computer 90, 92 contains system bus 98, where a bus is a set of hardware lines used for data transfer among the components of a computer or processing system. Bus 98 is essentially a shared conduit that connects different elements of a computer system (e.g., processor, disk storage, memory, input/output ports, network ports, etc.) that enables the transfer of information between the elements. Attached to system bus 98 is I/O device interface 100 for connecting various input and output devices (e.g., keyboard, mouse, displays, printer, speakers, etc.) to the computer 90, 92. Network interface 104 allows the computer to connect to various other devices attached to a network (e.g., network 94 of FIG. 9). Memory 106 provides volatile storage for computer software instructions 108 and data 110 used to implement an embodiment of the present invention (e.g., annotated Rose model and model interpreter EMF code). Disk storage 112 provides non-volatile storage for computer software instruction 108 and data 110 used to implement an embodiment of the present invention. Central processor unit 102 is also attached to system bus 98 and provides for the execution of computer instructions.

A computer readable program can, for example, include computer useable program code that causes a computer to design PNA-DNA oligomers (e.g., forward and reverse primers) and a probe that can be used with the PNA-DNA oligomer (e.g., PrimerProbeSet) in, for example, real-time PCR. The computer useable program code can be written in the following way:
Definition is called when PrimerProbeSet class has been created, in order to initialize the object;
Variables are organized and initialized by definition
   PercentGC
   MeltingTemp
   NucleotideRepeats
   PNAPurine
   PNAPercentGC
   PNAMeltingTemp
   PNANucleotideRepeats
   ProbePercentGC
   ProbeMeltingTemp
   ProbeNucleotideRepeats
   ProbeBeginG
   ReversePercentGC
   ReverseMeltingTemp
   ReverseNucleotideRepeats
   ReversePNAPurine
   ReversePNAPercentGC
   ReversePNAMeltingTemp
   ReversePNANucleotideRepeats
   ReverseComplement;
Definition calculates the percent of C and G bases out of total bases (C, G, A, and T) in a given forward primer sequence,
   Forward primer sequences with CG percentages between 30% and 80% are acceptable;
Definition calculates the melting temperature of a given forward primer sequence using the following formula Tₘ=AT*2+CG*4,
   Forward primer sequences with melting temperatures between 54 and 64 degrees Celcius are acceptable;
Definition determines if a given forward primer sequence contains undesirable stretches of repeated nucleotides,
   Forward primer sequences with the following repeats are NOT acceptable, AAAA, TTTT, CCC or GGG;
Definition calculates the percent of purine (A and G) in the PNA portion of a given forward primer sequence,
   Forward primer sequences with PNA purine content >=60% are NOT acceptable because PNA with high purine content tends to aggregate in solution;
Definition calculates the percent of C and G bases out of total bases (C, G, A and T) in the PNA portion of a given forward primer sequence,
   Forward primer sequences with PNA portions having GC percentages between 30% and 80% are acceptable;
Definition calculates the melting temperature of the PNA portion of a given forward primer sequence using the following formula Tₘ=AT*2+GC*4,
   Forward primer sequences with PNA portions having melting temperatures between 9 and 15 degrees Celcius are acceptable;
Definition determines if the PNA portion of a given forward primer sequence contains undesirable stretches of repeated nucleotides,
   Forward primer sequences with PNA portions containing the following repeats are NOT acceptable CCC or GGG;
Definition calculates the percent of C and G bases out of total bases (C, G, A and T) in a given probe sequence,
   Probe sequences with GC percentages between 30% and 80% are acceptable;
Definition calculates the melting temperature of a given probe sequence using the following formula Tₘ=AT*2+GC*4,
   Probe sequences with melting temperatures between 64 and 85 degrees Celcius are acceptable;
Definition determines if a given probe sequence contains undesirable stretches of repeated nucleotides,
   Probe sequences with the following repeats are NOT acceptable AAAA, TTTT, CCC or GGG
Definition determines if a given probe sequence begins with a G,
   Probe sequences with a 5′ G (those that begin with a G) are NOT acceptable;
Definition calculates the percent of C and G bases out of total bases (C, G, A, and T) in a given reverse primer sequence,
   Reverse primer sequences with CG percentages between 30% and 80% are acceptable;
Definition calculates the melting temperature of a given reverse primer sequence using the following formula Tₘ=AT*2+CG*4
   Reverse primer sequences with melting temperatures between 54 and 64 degrees Celcius are acceptable;
Definition determines if a given reverse primer sequence contains undesirable stretches of repeated nucleotides,
   Reverse primer sequences with the following repeats are NOT acceptable, AAAA, TTTT, CCC or GGG;
Definition calculates the percent of purine (A and G) in the PNA portion of a given reverse primer sequence,
   Reverse primer sequences with PNA purine content >=60% are NOT acceptable because PNA with high purine content tends to aggregate in solution;
Definition calculates the percent of C and G bases out of total bases (C, G, A and T) in the PNA portion of a given reverse primer sequence,
   Reverse primer sequences with PNA portions having GC percentages between 30% and 80% are acceptable;
Definition calculates the melting temperature of the PNA portion of a given reverse primer sequence using the following formula Tₘ=AT*2+GC*4,
   Reverse primer sequences with PNA portions having melting temperatures between 9 and 15 degrees Celcius are acceptable;
Definition determines if the PNA portion of a given reverse primer sequence contains undesirable stretches of repeated nucleotides,
   Reverse primer sequences with PNA portions containing the following repeats are NOT acceptable CCC or GGG.

Furthermore, in a preferred embodiment, the PNA-DNA oligomers and/or probes have minimal intra- and inter-molecular binding. Intra-molecular binding (self-complementary sequence) can lead to the formation of secondary structures, such as hairpins, or can lead to the extension and amplification of the PNA-DNA oligomers and/or probes themselves, instead of the amplification of target nucleic acid. PNA-DNA oligomer/probe sets with inter-molecular binding (complementary sequence shared between the oligomer and the probe) can lead to decreased reaction efficiency and to the formation of undesired product.

### EXEMPLIFICATION

### Example 1 Real-Time Quantitative Polymerase Chain Reaction (QPCR)

Amplification efficiency varies greatly during the early cycles of polymerase chain reaction (PCR) and improvement of early stage amplification yields an earlier cycle threshold value. Primer binding, primer stability and the number of fully extended PCR products governs the success of PCR. A number of factors, including the stability of primer-nucleic acid binding and PCR reagent concentrations (e.g., DNA polymerase, primers, MgCl₂) control primer binding. Consequently, increasing the thermal stability of primers and reducing their susceptibility to degradation may improve early stage amplification by improving the efficiency of PCR.

Experimental Design. PNA-DNA oligomers and DNA primers were tested under a wide range of standard PCR conditions to determine their relative efficiency in amplifying target Bacillus anthracis DNA. The virulence plasmid of Bacillus anthracis (Anthrax), pX01 was chosen as the target DNA for the PNA-DNA oligomer primers. Purposeful release of small amounts of Anthrax into the environment could pose significant health risks. Identification of Anthrax in environmental samples without the need for sample preparation would further increase the speed of Anthrax detection, particularly using real-time PCR (QPCR) detection, improving response time and situational awareness for first responders.

Traditional QPCR reactions have been optimized for the amplification of target DNA by DNA primer/probe sets and have therefore not been optimized for the use of PNA-DNA oligomers/probe sets. These experiments were completed by adding a known concentration of target DNA to QPCR reagents prepared with a fluorescent probe for the detection of product accumulation using either the PNA-DNA oligomers or DNA primers. Each PNA-DNA oligomer/probe set was assayed in triplicate for at least two dilutions of target (1 ug/mL to 0.01 ug/mL) and their average cycle threshold value compared to that of the corresponding DNA primer/probe set. Negative controls, consisting of QPCR reagents and no target DNA were included in QPCR to make sure that any successful QPCR amplification was not due to reagent contamination. Successful PNA-DNA oligomers were defined as those that have an equivalent or lower cycle threshold value than the corresponding DNA primers.

Conditions were varied to determine the optimal PNA-DNA oligomer concentrations (300-3000 nM), annealing (45-55° C.) and extension (65-72° C.) temperatures, number of amplification cycles (40-150), and salt concentration (2-6 mM MgCl₂). In addition, PNA-DNA oligomers with different PNA moiety lengths (1 or 3 PNA bases) directed towards the protective antigen (PA) gene in the pX01 virulence plasmid of Bacillus anthracis were included in the experiments to ascertain the effect of PNA length on QPCR signal. Both PNA-DNA oligomers and DNA primers were assayed to determine whether Taq polymerase would extend the PNA-DNA oligomers designed and whether the amplification was comparable to that seen with standard QPCR reagents.

Methods. The Bacillus anthracis PA gene sequence was located using GenBank® and the Bacillus anthracis (Ba) pX01 plasmid prepared by plasmid purification. Specifically, 50 mL of bovine heart infusion (BHI) were inoculated with a sample of Ba frozen vegetative cell stock, grown overnight at 37° C., with constant shaking at 300 rpm, and purified by using BioRad plasmid midiprep. The experiments required the design of unique PNA-DNA oligomer/probe sets. The probe contained 6-carboxy-fluorescein (FAM) as the fluorescent reporter dye at the 5′ terminus, and 6-carboxy-tetramethylrhodamine (TAMRA) as the quencher dye covalently linked to the 3′ terminus. Determining whether any other organisms have identical PNA-DNA oligomer/probe binding sequences required a FastA search (Pearson WR, PNAS 85(8):2444-2448, 1988) in the EMBL prokaryotic database.

Stratagene™ MX3000 real-time PCR system performed the cycling conditions necessary for the QPCR reactions. Each 50 μL optimized reaction contained 1× Stratagene™ Brilliant QPCR master mix (used according to the manufacturer's instructions), 300 nM of each primer, 200 nM fluorescent probe, and 30 nM reference dye. Other than the negative controls, each reaction also contained 1 ng of pX01 Bacillus anthracis plasmid DNA. All assay reactions were added in triplicate to a 96-well plate. The cycle threshold (CT), or the PCR cycle at which fluorescence first occurs, was determined automatically by using the sequence detector software (MX3000P; Stratagene™).

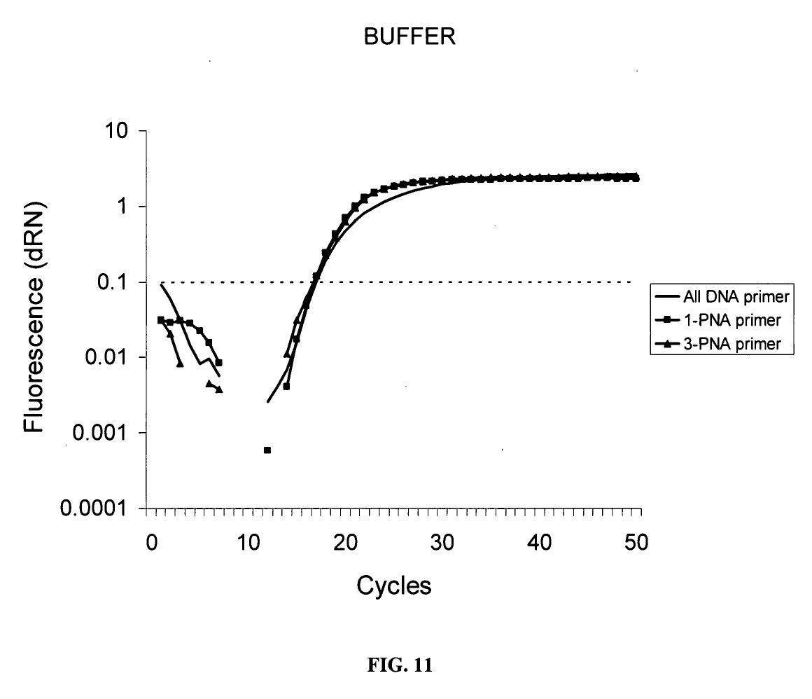

Results. PNA-DNA oligomers were able to be extended by Taq and support PCR amplification of target DNA (B. anthracis protective antigen gene). The PNA-DNA oligomers were found to be equivalent to and as effective as primers comprised of all DNA (see, e.g., FIG. 11) in pristine conditions. The conditions that proved successful with all primers were a primer concentration of 300 nM, a 55° C. annealing and 72° C. extension temperatures, 50 amplification cycles, and 5.5 mM MgCl₂.); however, other similar conditions (e.g., annealing temperatures from 50° C.-60° C.) are likely to also be successful.

### Example 2 QPCR Under Different Sample Conditions

Rapid identification and quantification of DNA in environmental samples is difficult at best. Primer binding in the presence of salts and primer stability in the presence of enzymes that degrade DNA limit successful detection using QPCR. PNA-DNA oligomers that are less dependent on ionic strength for binding to template DNA strands than DNA primers and are stable in the presence of enzymes. Faster thermal cycling would have a great impact on the speed of product detection using QPCR, an important step in reaching the ultimate goal of instant quantitative product detection.

Experimental design. A known concentration of target DNA was added to varying dilutions of experimental samples to ascertain the effect of environmental inhibitors on target DNA detection by PNA-DNA oligomers and DNA primers. Even at high dilutions, environmental samples often contain sufficient inhibitors to eliminate QPCR amplification of target DNA. DNA primers and fluorescent probe amplification of target DNA in environmental samples served as a positive control, indicating the effectiveness of standard QPCR reagents to amplify target DNA in non-ideal conditions. Each PNA-DNA oligomer/probe set was assayed in triplicate and their average cycle threshold value compared to that of the corresponding DNA primer/probe set. A reproducible cycle threshold value less than the cycle threshold value of the corresponding DNA primer/probe set defined successful amplification of target DNA by PNA-DNA oligomers. PNA-DNA oligomers or DNA primers with DNA fluorescent probe in pristine conditions were also included in the experiments as positive controls. Additionally, negative controls, consisting of QPCR reagents and no target DNA were included in QPCR to make sure that any successful QPCR amplification is not due to reagent contamination.
Methods. Collected from the environment surrounding the laboratory, 30 grams of soil were re-suspended in 30 mL of nuclease-free PCR-grade water. Working solutions of the soil were prepared the day of QPCR by serially diluting the stock, 1 gram/mL soil, to the following g/mL concentrations in nuclease-free PCR-grade water: 1, 0.1, 0.01, and 0.001.

Bacillus anthracis does not naturally exist in large enough quantities in the environment for detection using QPCR. Thus, 1 μg/mL, final concentration, of pX01 was added to each soil dilution in order to determine the effect of inhibitors on target DNA detection. Each dilution of soil was assayed in triplicate. All assay reactions were prepared in 50 μL volumes and dispensed into a 96-well reaction plate and cycled on the Stratagene™ MX3000 real-time PCR system as described previously. Plate cycling parameters were as follows: 1 cycle at 95° C. for 10 minutes, and 40 cycles each consisting of the following 95° C. for 30 seconds, 55° C. for 1 minute, and 72° C. for 30 seconds. Stratagene™ MX3000P sequence detector software was used to analyze all of the data.

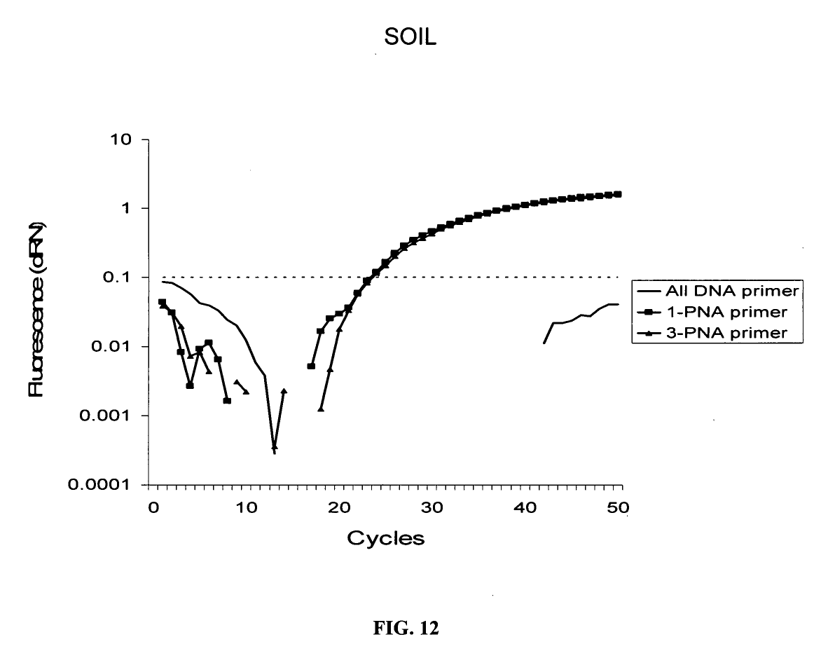

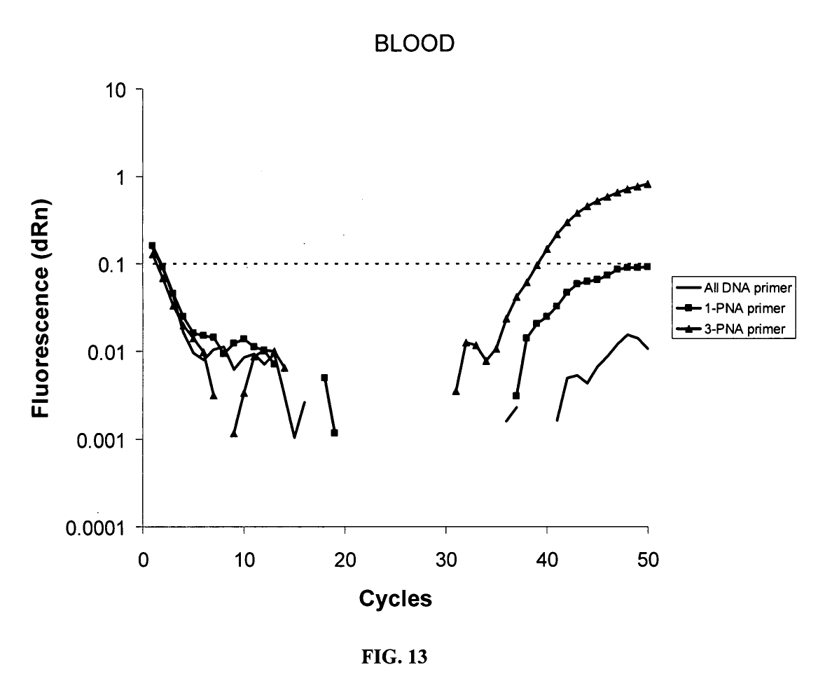

Results. The binding of PNA-DNA oligomers to target DNA irrespective of ionic strength and their stability in the presence of enzymes were predicted make PNA-DNA oligomers more suitable primers for environmental QPCR than DNA primers. In pristine conditions, chimeric primers consisting of 1 and 3 PNA bases demonstrated equivalent amplification of target DNA when compared with traditional DNA primers. (see FIG. 11). However, in both soil and blood samples, detection of B. anthracis DNA was observed with the PNA-DNA oligomer/probe set at target concentrations. In these challenging matrices, (e.g., soil and blood samples), the PNA-DNA oligomers were found to either be equivalent to or improve upon the detection of the DNA, depending on the concentration of inhibitors in each sample. Specifically, in inhibitory samples, the cycle threshold (CT) of the PNA-DNA oligomers was shifted slightly later than the corresponding signal in pristine conditions. However, the all-DNA primers had a CT later than their corresponding PNA-DNA primers (e.g., PAGIID4 compared to PAGIIP4). In some cases, the all-DNA primers did not afford any positive signal in QPCR. FIGS. 12 and 13 show the fluorescent light output from real-time PCR as a function of amplification CT for soil and blood, respectively.

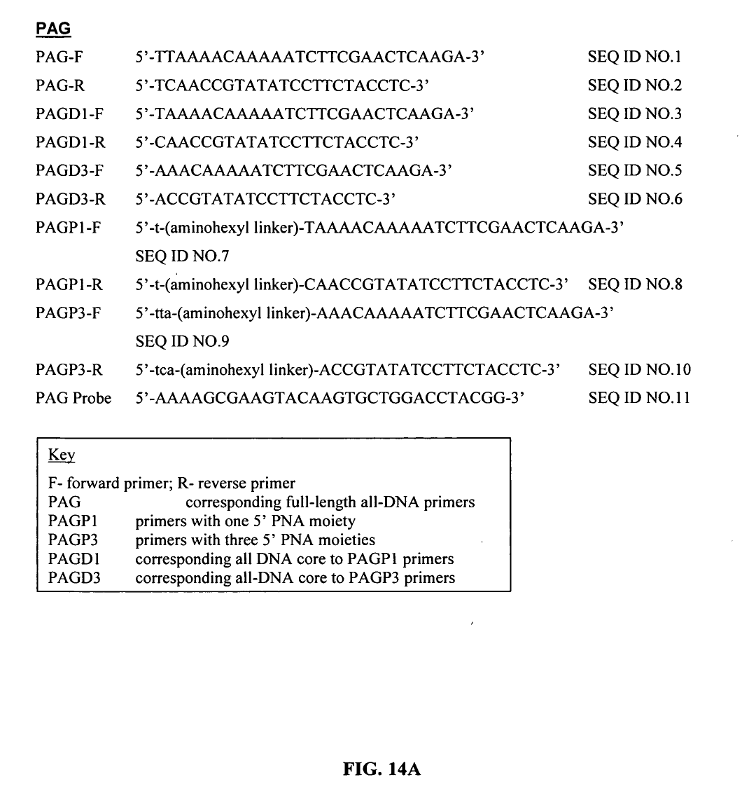

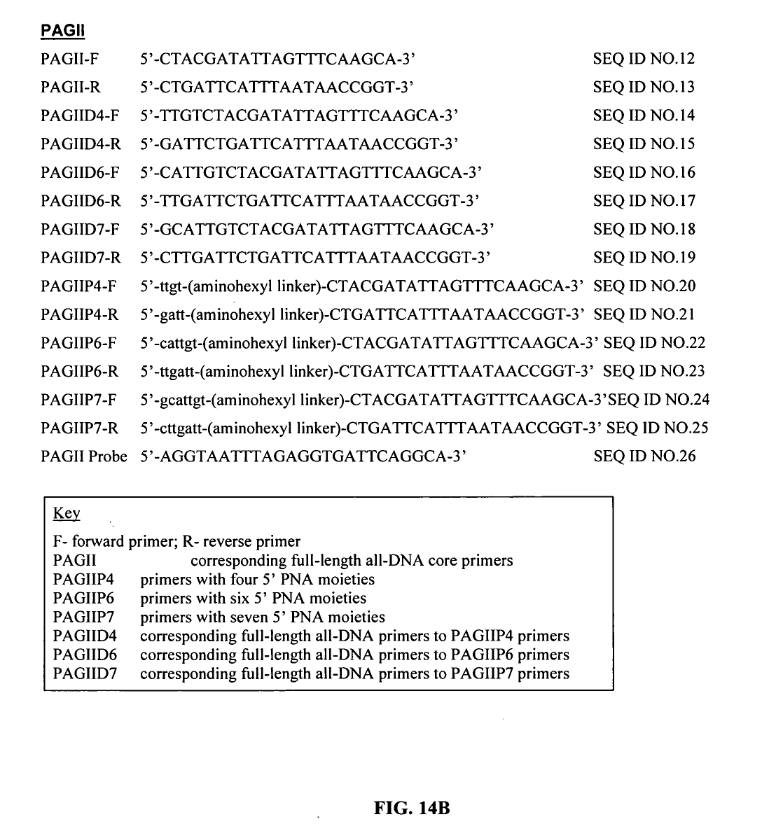

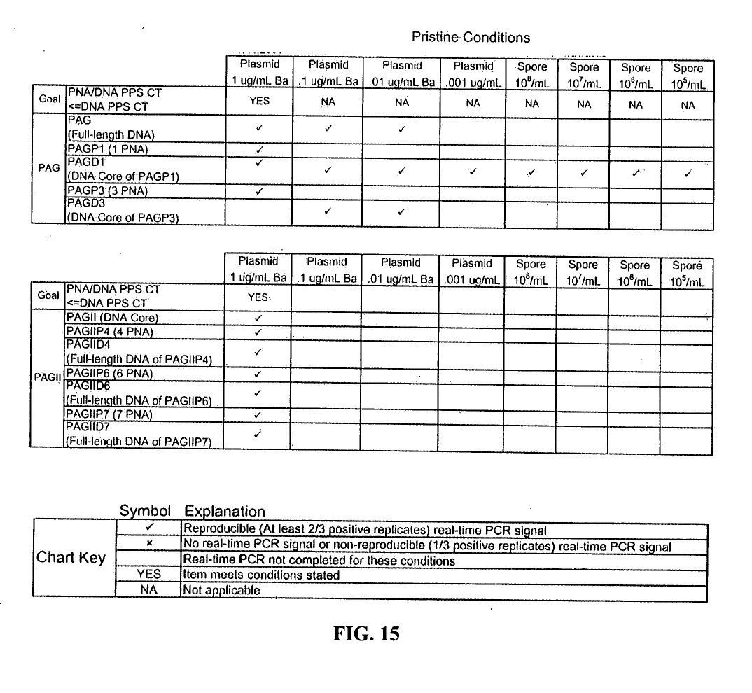

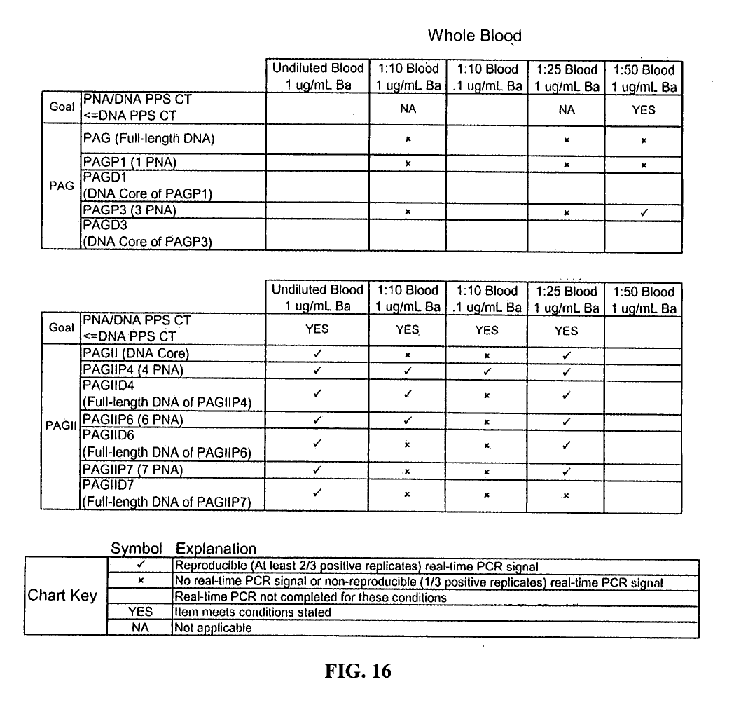

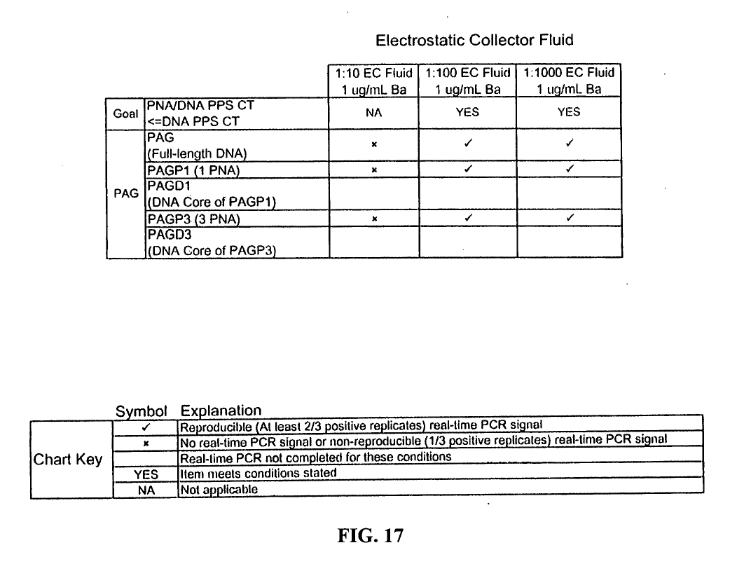

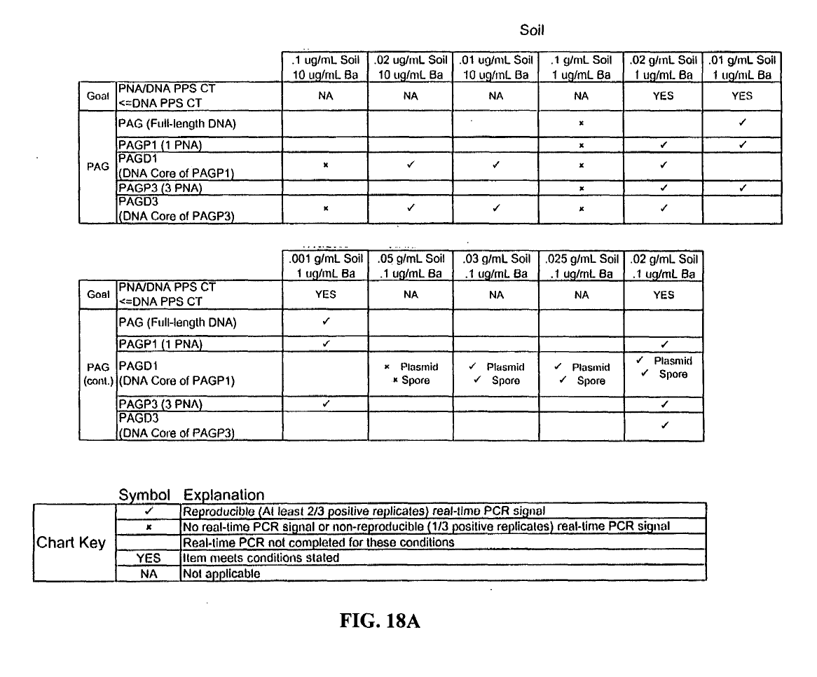

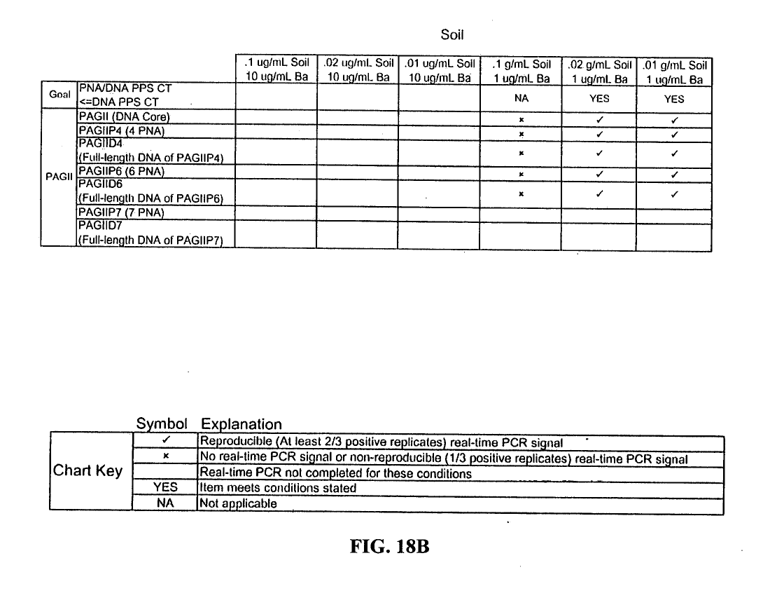

In addition, PNA-DNA oligomers of different sequences (see FIGS. 14A-14B) were comprehensively tested under a number of different sample conditions to determine the effect of oligomer sequence variation with respect to supporting PCR in challenging matrices. FIGS. 15-18 demonstrate the effectiveness of the various PNA-DNA oligomers in pristine (FIG. 15), whole blood (FIG. 16), electrostatic collector fluid (FIG. 17) and soil samples (FIGS. 18A-18B).

### Example 3 PNA-DNA Oligomer Design

Unlike the case for DNA primer design, no commercially available programs exist for PNA/DNA chimeric primer design. A computer program was written in MATLAB that locates forward and reverse PNA-DNA oligomer binding sites. From the possible forward and reverse PNA binding sites, a PNA-DNA oligomer and probes were selected by hand from 181,677 base pairs of the B. anthracis plasmid, pXO1 by searching for sequences matching the aforementioned constraints.

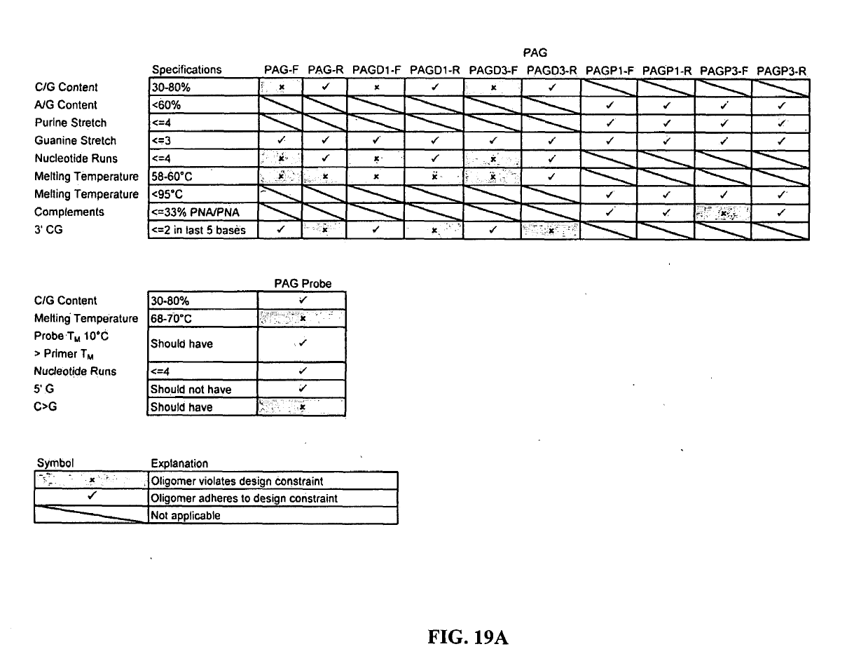

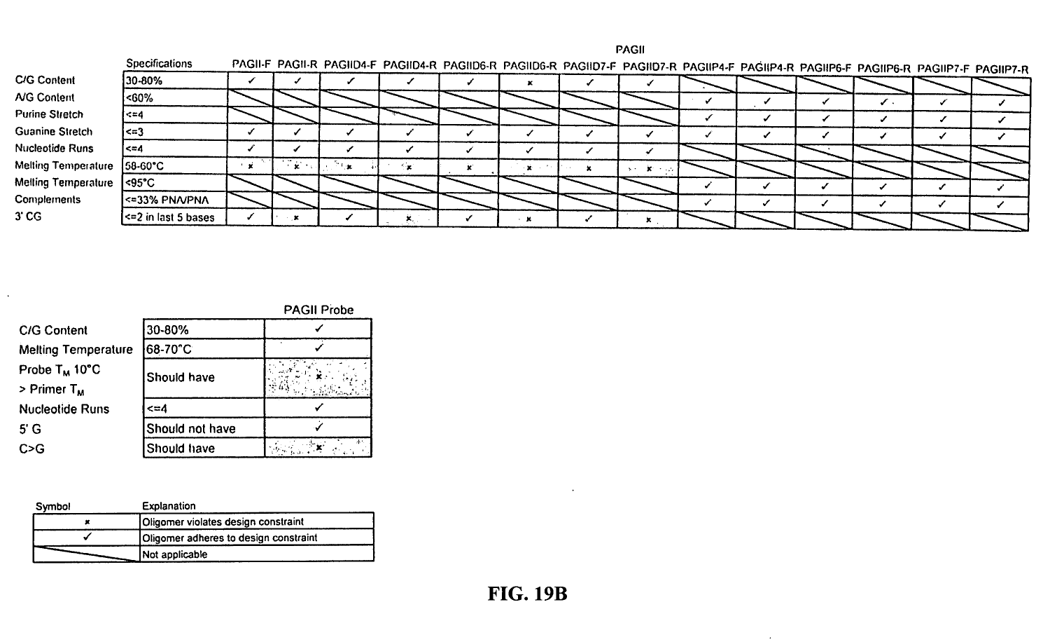

Based on experiments performed with PNA-DNA oligomers and labeled probes in QPCR (see FIGS. 19A and 19B), parameters for identifying and/or optimizing PNA-DNA oligomers and/or DNA probes for a target nucleic acid were developed. The parameters of the algorithm are as follows:

### PNA-DNA Oligomers (Forward and Reverse PCR Primers)

Guanine (G) and cytosine (C) nucleotide content (GC content) of between 30% and 80%.
Melting temperature of between 54° C. and 64° C.
Adenine (A) and thymine (T) nucleotide repeats of less than 4 bases; G and C nucleotide repeats of less than 3 bases.
Purine content (percent of adenine (A) and guanine (G) nucleotides) of PNA portion of less than 60%.
Melting temperature of PNA portion between 9° C. and 15° C.
No nucleotide repeats in the PNA portion of CCC or GGG.

### Probe

GC content of between 30% and 80%.
Melting temperature of between 65° C. and 85° C.
A and T nucleotide repeats of less than 4 bases; G and C nucleotide repeats of less than 3 bases.
First nucleotide base can not be a G.
   In addition, the sequence of the potential PNA-DNA oligomer and potential probe should be checked for intra-complementary (self-binding) sequence and inter-complementary (oligomer-probe-binding) sequence, and any such sequence eliminated or minimized.

The relevant teachings of all references, patents and patent applications cited herein are incorporated herein by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A PNA-DNA oligomer comprising: a peptide nucleic acid (PNA) oligomer and a deoxyribonucleic acid (DNA) oligomer, wherein the PNA oligomer and DNA oligomer are covalently linked by a C₆ amino linker having the formula:

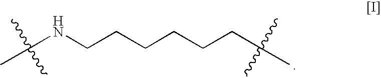

2. The PNA-DNA oligomer of claim 1, wherein the PNA is about 1 to about 7 bases.

3. A method for detecting the presence of a target nucleic acid in a sample comprising:
a) combining a peptide nucleic acid (PNA)-deoxyribonucleic acid (DNA) oligomer, a labeled probe, a DNA polymerase having exonuclease activity, unlabeled deoxyribonucleotide triphosphates (dNTPs) and said sample, thereby forming a combination;
b) maintaining said combination under conditions suitable for extending said PNA-DNA oligomer in the presence of the target nucleic acid wherein:
i) said PNA-DNA oligomer and labeled probed are annealed to said target nucleic acid and wherein said labeled probe anneals to the target nucleic acid downstream of the PNA-DNA oligomer; and
ii) said DNA polymerase extends said PNA-DNA oligomer annealed to the target nucleic acid in the presence of the unlabeled deoxyribonucleotide triphosphates (dNTPs), thereby forming a full-length unlabeled nucleic acid product, wherein the exonuclease activity of the DNA polymerase degrades the labeled probe annealed to the target nucleic acid during extension of the PNA-DNA oligomer annealed to the target nucleic acid, thereby resulting in emission of a detectable signal; and
c) analyzing said combination for emission of said detectable signal, wherein emission of the detectable signal indicates the presence of the target nucleic acid in the sample.

4. The method of claim 3, wherein the labeled probe is fluorescently labeled with a high energy dye on the 5′ end of the probe and a low energy dye on the 3′ end of the probe in a fluorescent resonance energy transfer (FRET) format.

5. The method of claim 4, wherein the labeled probe is at least about 17 bases long and anneals between about 1 to about 5 bases downstream of the PNA-DNA oligomer.

6. The method of claim 3, wherein the method further comprises amplifying said full-length unlabeled nucleic acid product.

7. The method of claim 3, wherein the PNA-DNA oligomer is comprised of a PNA oligomer and a DNA oligomer, wherein the PNA oligomer and DNA oligomer are covalently linked by a C₆ amino linker having the formula:

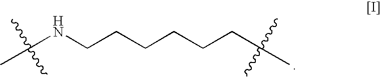

8. The method of claim 3, wherein the PNA-DNA oligomer has a purine content of less than about 60%, a guanine-cytosine (GC) content of between about 30% to about 80% and single nucleotide repeats of less than about 4 bases.

9. The method of claim 3, wherein said sample is selected from the group consisting of a biological sample, an environmental sample, a contaminated sample, a suboptimal sample, a substantially purified sample and a pristine sample.

10. The method of claim 9, wherein said biological sample is selected from the group consisting of muscous, saliva, urine, feces, whole blood, plasma, serum, cerebrospinal fluid, alveolar lavages, sweat, tears, a carcass sample, a nasal swab sample and combinations thereof.

11. The method of claim 9, wherein said environmental sample is selected from the group consisting of water, soil, air, sewage, food, crops, plant tissue, surface wipes, forensic samples and combinations thereof.

12. The method of claim 9, wherein said suboptimal sample is selected from the group consisting of a bacterial culture supernatant, a food sample, a sample with a high pH or a low pH, a sample with a high salt concentration or a low salt concentration, a sample with inhibitory proteins, a sample with heavy metals and combination thereof.

13. The method of claim 3, wherein emission of said detectable signal indicates the presence of a target nucleic acid of one or more microorganisms.

14. The method of claim 13, wherein said one or more microorganisms is a pathogen.

15. A method for detecting the presence of a target ribonucleic acid (RNA) in a sample comprising:
a) combining a first peptide nucleic acid (PNA)-deoxyribonucleic acid
(DNA) oligomer, a reverse transcriptase enzyme, unlabeled deoxyribonucleotide phosphates (dNTPs) and said sample, thereby forming a first combination;
b) maintaining said combination under conditions suitable for extending said first PNA-DNA oligomer in the presence of the target RNA wherein:
i) said first PNA-DNA oligomer anneals to said target RNA in said sample; and
ii) said reverse transcriptase enzyme extends said first PNA-DNA oligomer annealed to the target RNA in the presence of the unlabeled dNTPs, thereby forming a full-length unlabeled cDNA product;
c) combining a second PNA-DNA oligomer, a labeled probe, a DNA polymerase having exonuclease activity and the full-length unlabeled cDNA product, thereby forming a second combination;
d) maintaining said second combination under conditions suitable for extending said second PNA-DNA oligomer in the presence of the full-length unlabeled cDNA product wherein:
i) said second PNA-DNA oligomer and labeled probed are annealed to the full-length unlabeled cDNA product and wherein said labeled probe anneals to the full-length unlabeled cDNA product downstream of the second PNA-DNA oligomer; and
ii) said DNA polymerase extends said second PNA-DNA oligomer annealed to said full-length unlabeled cDNA product in the presence of the unlabeled dNTPs, thereby forming a full-length unlabeled DNA product, wherein the exonuclease activity of the DNA polymerase degrades the labeled probe annealed to the full-length unlabeled cDNA product during extension of the second PNA-DNA oligomer annealed to the full-length unlabeled cDNA product, thereby resulting in emission of a detectable signal; and
e) analyzing said second combination for emission of said detectable signal, wherein emission of said detectable signal indicates the presence of said target RNA in the sample.

16. A method for detecting the presence of a target nucleic acid in a sample comprising:
a) combining a peptide nucleic acid (PNA)-deoxyribonucleic acid (DNA) oligomer, a DNA polymerase, unlabeled deoxyribonucleotide triphosphates (dNTPs), and said sample, thereby forming a combination;
b) maintaining said combination under conditions suitable for extending said PNA-DNA oligomer in the presence of the target nucleic acid wherein:
i) said PNA-DNA oligomer is annealed to said target nucleic acid; and
ii) said DNA polymerase extends said PNA-DNA oligomer annealed to the target nucleic acid in the presence of the unlabeled dNTPs, thereby forming a full-length unlabeled target nucleic acid product;
c) amplifying said full-length unlabeled target nucleic acid product comprising:
i) denaturing the full-length unlabeled target nucleic acid product,
ii) maintaining the PNA-DNA oligomer, the DNA polymerase, a reverse complementary primer and the denatured full-length unlabeled target nucleic acid product under conditions suitable for extending the PNA-DNA oligomer and said reverse complementary primer in the presence of the denatured full-length unlabeled target nucleic acid product, wherein the PNA-DNA oligomer and the reverse complementary primer anneal to the denatured full-length unlabeled target nucleic acid product and wherein the DNA polymerase extends the PNA-DNA oligomer and the reverse complementary primer annealed to the denatured full-length unlabeled target nucleic acid product in the presence of unlabeled dNTPs, thereby forming full-length unlabeled target nucleic acid product, and
iii) repeating steps i) and ii) one or more times, thereby producing one or more full-length unlabeled target nucleic acid products; and
d) detecting said one or more full-length unlabeled target nucleic acid products,
wherein the presence of one or more full-length target nucleic acid products indicates the presence of said target nucleic acid in the sample.

17. The method of claim 16, wherein said one or more full-length unlabeled target nucleic acid products are detected by a DNA-intercalating agent or a dye that preferentially binds double-stranded DNA.

18. A method of amplifying a target nucleic acid comprising:
a) combining a peptide nucleic acid (PNA)-deoxyribonucleic acid (DNA) oligomer, a reverse complementary primer, a labeled probe, a DNA polymerase having exonuclease activity, unlabeled deoxyribonucleotide triphosphates (dNTPs) and said target nucleic acid, thereby forming a combination;
b) maintaining said combination under conditions suitable for extending said PNA-DNA oligomer in the presence of the target nucleic acid wherein:
i) said PNA-DNA oligomer and labeled probed are annealed to said target nucleic acid and wherein said labeled probe anneals to the target nucleic acid downstream of the PNA-DNA oligomer; and
ii) said DNA polymerase extends said PNA-DNA oligomer annealed to the target nucleic acid in the presence of the unlabeled dNTPs, thereby forming full-length unlabeled target nucleic acid product, wherein the exonuclease activity of the DNA polymerase degrades the labeled probe annealed to the target nucleic acid during extension of the PNA-DNA oligomer annealed to the target nucleic acid, thereby resulting in emission of a detectable signal;
c) denaturing the full-length unlabeled target nucleic acid product and;
d) repeating steps b) and c) one or more times, thereby forming one or more unlabeled target nucleic acid products and thereby amplifying the target nucleic acid.

19. The method of claim 18 wherein said target nucleic acid is denatured prior to the annealing of said PNA-DNA oligomer, said reverse complementary primer and said labeled probe to said target nucleic acid.

20. The method of claim 18 further comprising detecting the emission of said detectable signal, thereby detecting the amplification of the target nucleic acid in real time.

21. The method of claim 18, wherein the PNA-DNA oligomer is comprised of a PNA oligomer and a DNA oligomer, wherein the PNA oligomer and DNA oligomer are covalently linked by a C₆ amino linker having the formula:

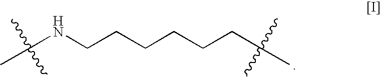

22. The method of claim 18 wherein the reverse complementary primer is a second PNA-DNA oligomer.

23. A method of amplifying a target nucleic acid comprising:
a) combining a peptide nucleic acid (PNA)-deoxyribonucleic acid (DNA) oligomer, a reverse complementary primer, a DNA polymerase, unlabeled deoxyribonucleotide triphosphates (dNTPs) and said target nucleic acid, thereby forming a combination;
b) maintaining the PNA-DNA oligomer, the DNA polymerase, a reverse complementary primer and the target nucleic acid, under conditions suitable for extending the PNA-DNA oligomer and said reverse complementary primer in the presence of the target nucleic acid, wherein:
i) the PNA-DNA oligomer and the reverse complementary primer anneal to the target nucleic acid, and
ii) the DNA polymerase extends the PNA-DNA oligomer and the reverse complementary primer annealed to the target nucleic acid, in the presence of the unlabeled dNTPs, thereby forming full-length unlabeled target nucleic acid product;
c) denaturing the full-length unlabeled target nucleic acid product; and
d) repeating steps b) and c) one or more times, thereby forming one or more full-length unlabeled target nucleic acid products and thereby amplifying the target nucleic acid.

24. The method claim 23 wherein said target nucleic acid is denatured prior to the annealing of said PNA-DNA oligomer and said reverse complementary primer to said target nucleic acid.

25. The method of claim 23 wherein said PNA-DNA oligomer comprises a PNA oligomer and a DNA oligomer, wherein the PNA oligomer and DNA oligomer are covalently linked by a C₆ amino linker having the formula:

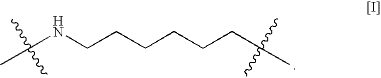

26. The method of claim 23 wherein the reverse complementary primer is a second PNA-DNA oligomer.

27. A method of probing a target nucleic acid comprising:
a) hybridizing to the target nucleic acid an unlabeled peptide nucleic acid (PNA)-deoxyribonucleic acid (DNA) oligomer comprising at least one PNA oligomer and at least one DNA oligomer, wherein the at least one PNA oligomer and at least one DNA oligomer are covalently linked by a linker selected from the group consisting of a C₆ amino linker having the formula: 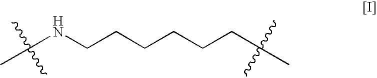
and a C₅ carboxy linker having the formula: 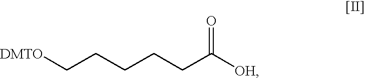
wherein DMTO is 3,5-dimethyl-1,2,4-trioxolane; and
b) detecting the hybridized unlabeled PNA-DNA oligomer.

28. The method of claim 27, wherein said hybridized unlabeled PNA-DNA oligomer is detected by the cyanine dye 3,3′-diethylthiadicarbocyanine iodide (DiSc₂(5)).

29. The method of claim 27, wherein said target nucleic acid is selected from the group consisting of synthetic nucleic acid, genetically engineered nucleic acid, genomic DNA, chromosomal DNA, mitochondrial DNA, transposon DNA, plasmid DNA, synthetic, RNA, ribosomal RNA, viral RNA and combinations thereof.

30. The method of claim 27, wherein the method is for detecting one or more nucleic acid changes selected from the group consisting of a mutation of, amplification of, addition to and deletion in said target nucleic acid.

31. A method for designing a peptide nucleic acid (PNA)-deoxyribonucleic acid (DNA) oligomer that binds a target nucleic acid comprising:
a) obtaining the sequence of the target nucleic acid; and
b) determining a complementary PNA-DNA oligomer sequence for a region on said target nucleic acid, thereby identifying a potential PNA-DNA oligomer, wherein the potential PNA-DNA oligomer is accepted or rejected in a method comprising:
i) calculating the percent of guanine (G) and cytosine (C) nucleotides in the potential PNA-DNA oligomer, wherein if said percent of G and C nucleotides is between about 30% and about 80%, then the potential PNA-DNA oligomer is accepted;
ii) calculating the melting temperature of the potential PNA-DNA oligomer, wherein if the melting temperature is between about 54° C. and about 64° C., then the potential PNA-DNA oligomer is accepted;
iii) determining the number of contiguous adenine (A), contiguous thymine (T), contiguous guanine (G) and contiguous cytosine (C) nucleotides in the potential PNA-DNA oligomer, wherein if there are less than: (a) four contiguous A nucleotides, (b) four contiguous T nucleotides, (c) three contiguous C nucleotides and (d) three contiguous G nucleotides, then the potential PNA-DNA oligomer is accepted;
iv) calculating the percent of adenine (A) and guanine (G) nucleotides in the PNA oligomer portion of the potential PNA-DNA oligomer, thereby determining the purine content of the potential PNA-DNA oligomer, wherein if said percent of A and G nucleotides is less than or equal to about 60%, then the potential PNA-DNA oligomer is accepted;
v) calculating the percent of guanine (G) and cytosine (C) nucleotides in the PNA oligomer portion of the potential PNA-DNA oligomer, wherein if said percent of G and C nucleotides is between about 30% and about 80%, then the potential PNA-DNA oligomer is accepted;
vi) calculating the melting temperature of the PNA oligomer portion of the potential PNA-DNA oligomer, wherein if the melting temperature is between about 9° C. and about 15° C., then the potential PNA-DNA oligomer is accepted, and
vii) determining the number of contiguous G and C nucleotides in the
PNA oligomer portion of the potential PNA-DNA oligomer, wherein if there are not three contiguous G or three contiguous C nucleotides, then the PNA-DNA oligomer is accepted.

32. The method of claim 31 wherein the method is performed by a computer.

33. The method of claim 31, wherein the PNA-DNA oligomer is selected from the group consisting of a forward primer and a reverse primer.

34. The method of claim 31 further comprising designing a probe, the method further comprising:
a) determining a probe-binding region on said target nucleic acid located between 1 and 5 nucleotides 3′ to the region said PNA-DNA oligomer binds said target nucleic acid;
b) determining a complementary nucleotide sequence for said probe-binding region, thereby identifying a potential probe, wherein the potential probe is accepted or rejected in a method comprising:
i) determining a complementary nucleotide sequence to said probe-binding region, thereby identifying a potential probe;
ii) calculating the percent of cytosine (C) and guanine (G) nucleotides in said potential probe, wherein if the percent of C and G nucleotides is between about 30% and about 80%, then the potential probe is accepted;
iii) calculating the melting temperature for said potential probe, wherein if the melting temperature is between about 65° C. and about 85° C. then the potential probe is accepted;
iv) determining the number of contiguous adenine (A), contiguous thymine (T), contiguous guanine (G) and contiguous cytosine (C) nucleotides in the potential probe, wherein if there are less than: (a) four contiguous A nucleotides, (b) four contiguous T nucleotides, (c) three contiguous C nucleotides and (d) three contiguous G nucleotides, then the potential probe is accepted; and
v) identifying the first nucleotide base of the potential probe, wherein if the first nucleotide base is an adenine (A), thymine (T) or a cytosine (C), then the potential probe is accepted.

35. The method of claim 34 wherein the method is performed by a computer.

36. A computer program product comprising a computer useable medium including a computer readable program, wherein the computer readable program, when executed on a computer causes the computer to:
a) obtain the sequence of the target nucleic acid; and
b) determine a complementary peptide nucleic acid (PNA)-deoxyribonucleic acid (DNA) oligomer sequence for a region on said target nucleic acid, thereby identifying a potential PNA-DNA oligomer;
c) calculate the percent of guanine (G) and cytosine (C) nucleotides in the potential PNA-DNA oligomer, wherein if said percent of G and C nucleotides is between about 30% and about 80%, then the computer accepts the potential PNA-DNA oligomer;
d) calculate the melting temperature of the potential PNA-DNA oligomer, wherein if the melting temperature is between about 54° C. and about 64° C., then the computer accepts the potential PNA-DNA oligomer;
e) determine the number of contiguous adenine (A), contiguous thymine (T), contiguous guanine (G) and contiguous cytosine (C) nucleotides in the potential PNA-DNA oligomer, wherein if there are less than: (a) four contiguous A nucleotides, (b) four contiguous T nucleotides, (c) three contiguous C nucleotides and (d) three contiguous G nucleotides, then the computer accepts the potential PNA-DNA oligomer;
f) calculate the percent of adenine (A) and guanine (G) nucleotides in the PNA oligomer portion of the potential PNA-DNA oligomer, thereby determining the purine content of the potential PNA-DNA oligomer, wherein if said percent of A and G nucleotides is less than or equal to about 60%, then the computer accepts the potential PNA-DNA oligomer;
g) calculate the percent of guanine (G) and cytosine (C) nucleotides in the PNA oligomer portion of the potential PNA-DNA oligomer, wherein if said percent of G and C nucleotides is between about 30% and about 80%, then the computer accepts the potential PNA-DNA oligomer;
h) calculate the melting temperature of the PNA oligomer portion of the potential PNA-DNA oligomer, wherein if the melting temperature is between about 9° C. and about 15° C., then the computer accepts the potential PNA-DNA oligomer; and
i) determine the number of contiguous G and C nucleotides in the PNA oligomer portion of the potential PNA-DNA oligomer, wherein if there are not three contiguous G or three contiguous C nucleotides, then the computer accepts the potential PNA-DNA oligomer.

37. A computer program product comprising a computer useable medium including a computer readable program, wherein the computer readable program, when executed on a computer causes the computer to:
a) determine a probe-binding region on said target nucleic acid located between 1 and 5 nucleotides 3′ to the region a peptide nucleic acid (PNA)-deoxyribonucleic acid (DNA) oligomer binds a target nucleic acid;
b) determine a complementary nucleotide sequence for said probe-binding region, thereby identifying a potential probe;
c) calculate the percent of cytosine (C) and guanine (G) nucleotides in said potential probe, wherein if the percent of C and G nucleotides is between about 30% and about 80%, then the computer accepts the potential probe;
d) calculate the melting temperature for said potential probe, wherein if the melting temperature is between about 65° C. and about 85° C. then the computer accepts the potential probe;
e) determine the number of contiguous adenine (A), contiguous thymine (T), contiguous guanine (G) and contiguous cytosine (C) nucleotides in the potential probe, wherein if there are less than: (a) four contiguous A nucleotides, (b) four contiguous T nucleotides, (c) three contiguous C nucleotides and (d) three contiguous G nucleotides, then the computer accepts the potential probe and
f) identify the first nucleotide base of the potential probe, wherein if the first nucleotide base is an adenine (A), thymine (T) or a cytosine (C), then the computer accepts the potential probe.


SEQUENCE LISTING (26 sequences)

SEQ ID NO: 1 - DNA, 27 bp, Artificial Sequence
  Synthetic primer
ttaaaacaaaaatcttcgaactcaaga

SEQ ID NO: 2 - DNA, 23 bp, Artificial Sequence
  Synthetic primer
tcaaccgtatatccttctacctc

SEQ ID NO: 3 - DNA, 26 bp, Artificial Sequence
  Synthetic primer
taaaacaaaaatcttcgaactcaaga

SEQ ID NO: 4 - DNA, 22 bp, Artificial Sequence
  Synthetic primer
caaccgtatatccttctacctc

SEQ ID NO: 5 - DNA, 24 bp, Artificial Sequence
  Synthetic primer
aaacaaaaatcttcgaactcaaga

SEQ ID NO: 6 - DNA, 20 bp, Artificial Sequence
  Synthetic primer
accgtatatccttctacctc

SEQ ID NO: 7 - DNA, 27 bp, Artificial Sequence
  Synthetic primer
ttaaaacaaaaatcttcgaactcaaga

SEQ ID NO: 8 - DNA, 23 bp, Artificial Sequence
  Synthetic primer
tcaaccgtatatccttctacctc

SEQ ID NO: 9 - DNA, 27 bp, Artificial Sequence
  Synthetic primer
ttaaaacaaaaatcttcgaactcaaga

SEQ ID NO: 10 - DNA, 23 bp, Artificial Sequence
  Synthetic primer
tcaaccgtatatccttctacctc

SEQ ID NO: 11 - DNA, 30 bp, Artificial Sequence
  Synthetic primer
aaaagcgaagtacaagtgctggacctacgg

SEQ ID NO: 12 - DNA, 21 bp, Artificial Sequence
  Synthetic primer
ctacgatattagtttcaagca

SEQ ID NO: 13 - DNA, 21 bp, Artificial Sequence
  Synthetic primer
ctgattcatttaataaccggt

SEQ ID NO: 14 - DNA, 25 bp, Artificial Sequence
  Synthetic primer
ttgtctacgatattagtttcaagca

SEQ ID NO: 15 - DNA, 25 bp, Artificial Sequence
  Synthetic primer
gattctgattcatttaataaccggt

SEQ ID NO: 16 - DNA, 27 bp, Artificial Sequence
  Synthetic primer
cattgtctacgatattagtttcaagca

SEQ ID NO: 17 - DNA, 27 bp, Artificial Sequence
  Synthetic primer
ttgattctgattcatttaataaccggt

SEQ ID NO: 18 - DNA, 28 bp, Artificial Sequence
  Synthetic primer
gcattgtctacgatattagtttcaagca

SEQ ID NO: 19 - DNA, 28 bp, Artificial Sequence
  Synthetic primer
cttgattctgattcatttaataaccggt

SEQ ID NO: 20 - DNA, 25 bp, Artificial Sequence
  Synthetic primer
ttgtctacgatattagtttcaagca

SEQ ID NO: 21 - DNA, 25 bp, Artificial Sequence
  Synthetic primer
gattctgattcatttaataaccggt

SEQ ID NO: 22 - DNA, 27 bp, Artificial Sequence
  Synthetic primer
cattgtctacgatattagtttcaagca

SEQ ID NO: 23 - DNA, 27 bp, Artificial Sequence
  Synthetic primer
ttgattctgattcatttaataaccggt

SEQ ID NO: 24 - DNA, 28 bp, Artificial Sequence
  Synthetic primer
gcattgtctacgatattagtttcaagca

SEQ ID NO: 25 - DNA, 28 bp, Artificial Sequence
  Synthetic primer
cttgattctgattcatttaataaccggt

SEQ ID NO: 26 - DNA, 25 bp, Artificial Sequence
  Synthetic primer
aggtaatttagaggtgattcaggca